(19) United States
(12) Patent Application Publication
(10) Pub. No.: US 2010/0197564 A1
(43) Pub. Date: Aug. 5, 2010

(54) DIARYL MORPHOLINES AS CB1 MODULATORS

(75) Inventors: Jack D. Scott, Scotch Plains, NJ (US); Andrew Stamford, Chatham Township, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/593,777

(22) PCT Filed: Apr. 17, 2008
(86) PCT No.: PCT/US08/05000

§ 371 Date: Apr. 12, 2010

Related U.S. Application Data
(60) Provisional application No. 60/912,847, filed on Apr. 19, 2007.

(51) Int. Cl. A61K 31/5355; C07D 265/30; A61K 31/5375; C07D 413/12; A61K 38/28; A61K 31/397; A61P 3/00; A61P 3/04; A61P 3/06; A61P 25/00; A61P 29/00; A61P 1/00; A61P 9/00

(52) U.S. Cl. 514/3; 544/106; 514/239.5; 544/124; 514/237.2; 514/210.02

(57) ABSTRACT

The present invention provides compounds having the general structure of Formula (I) or a pharmaceutically acceptable salt, solvate, ester or prodrug thereof, which are useful as CB1 receptor antagonists. The compounds of the invention may be useful in treating diseases, disorders, or conditions responsive to CB1 receptor antagonists, including, but not limited to, metabolic syndrome, obesity, waist circumference, dyslipidemia, insulin sensitivity, neuroinflammatory disorders, cognitive disorders, psychosis, addictive behavior, gastrointestinal disorders, and cardiovascular conditions.

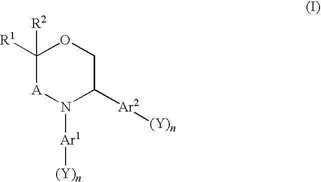

### FIELD OF THE INVENTION

The present invention relates to diaryl morpholines compounds useful as cannabinoid (“CB”) receptor antagonists, specifically, cannabinoid-1 receptor antagonists (“CB1 receptor antagonists”), to pharmaceutical compositions comprising such compounds, and to their use in treating conditions responsive to CB1 receptor antagonists. Non-limiting examples of such conditions include metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering of waist circumference, treatment of dyslipidemia, insulin sensitivity, insulin resistance, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome, and inflammatory bowel disease.

### BACKGROUND OF THE INVENTION

The CB1 receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB1 receptor antagonists, for example pyrazole derivatives such as rimonabant (e.g., U.S. Pat. No. 6,432,984), can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regul. Integr. Comp. Physiol. 2002 vol. 284, R343-R344), neuroinflammatory disorders (e.g., Adam, et al., Expert Opin. Ther. Patents, 2002, vol. 12, no. 10, 1475-1489; U.S. Pat. No. 6,642,258), cognitive disorders and psychosis (e.g., Adam et al., Expert Opin. Ther. Pat., 2002, vol. 12, pp. 1475-1489), addiction (e.g., smoking cessation; U.S. Patent Publ. 2003/0087933), gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60; Sanofi-Aventis Publication, Bear Stearns Conference, New York, Sep. 14, 2004, pages 19-24).

U.S. Patent Application Publication U.S. 2004/0167185 describes Edg-3 receptor inhibitors including substituted piperidines. U.S. Patent Application Publication U.S. 2002/0128476 and U.S. Patent Application Publication U.S. 2004/0180927 describe 3-piperidinone and 3-piperidinol cysteine protease inhibitors. U.S. Patent Application Publication U.S. 2001/0006972 describes aryl piperidine NK-1 receptor antagonists. U.S. Patent Application Publication U.S. 2003/0171588 describes piperidine-3-carboxamide derivatives. U.S. Pat. No. 5,234,895 describes 2-arylpyridone herbicides. U.S. Pat. No. 5,185,349 describes lactam ACAT inhibitors. U.S. Pat. No. 4,839,360 describes 1,6-diaryl-2-piperidones. U.S. Pat. No. 6,369,077 describes protease inhibitors. U.S. Pat. No. 5,332,817 describes 3-aminopiperidine derivatives. WO 03/062392 describes Edg-2, Edg-3, Edg-4, and Edg-7 antagonists. U.S. Pat. No. 5,580,883 describes piperidine derivatives which present nerve degeneration. U.S. Pat. No. 6,441,001 describes piperidine derivatives as CCR3 modulators. Weis et al., Tetrahedron, 59 (2003) 1403-1411, describe the synthesis of diphenylpyralines. Josephsohn et al., J. Am. Chem. Soc., 125 (2003) 4018-4019 describe methods of preparing diarylpiperidines. U.S. Pat. No. 5,719,147, discloses substituted heterocycles, including substituted morpholines, as tachykinin receptor antagonists. WO97/02824 discloses combinations of substituted heterocycles, including substituted morpholines, and muscarinic antagonists and/or antihistamine receptor antagonists for the treatment of motion sickness. WO96/20009 discloses substituted heterocycles, including substituted morpholines as tachykinin receptor angatonists in combination of opioid receptor antagonists for the treatment of pain. WO95/16679 discloses substituted morpholines and thiomorpholines as tachykinin receptor antagonists. EP577394 discloses substituted heterocycles, including substituted morpholines, as tachykinin receptor antagonists. WO2007/084450 discloses certain compounds as CB1 receptor angatonists. WO2006/060461 discloses certain diaryl piperidines as CB1 receptor antagonists. WO2006/039334 discloses certain CB1 receptor antagonists in combination with substituted azetidinones. And WO2005/087754 discloses substituted thiomorpholines and morpholines, including diaryl morpholines, as tachykinin receptor antagonists useful in treating inflammatory diseases, pain or migraine, or asthma. However, the compounds disclosed in each of the above references differ from the compounds of the present invention. There is still a need for improved cannabinoid agents, such as selective CB1 receptor antagonists, with good side-effect profiles and improved efficacy. It is therefore an object of the present invention to provide diaryl- and di-heteroaryl-substituted morpholine compounds, as well as single-agent and multi-agent compositions, useful in the treatment of diseases or conditions mediated by CB1 receptors.

### SUMMARY OF THE INVENTION

The present invention provides a novel class of substituted morpholine (and substituted morpholinone) compounds useful as CB1 receptor antagonists.

Thus, in one embodiment, the present invention provides a compound of Formula (I):

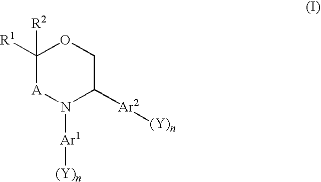

or a pharmaceutically acceptable salt, solvate, ester or prodrug thereof wherein Ar¹, Ar², R¹, R², A, each Y, and each n is selected independently and wherein:
Ar¹ and Ar² are each independently aryl or heteroaryl;
R¹ is alkyl, —(C(R³)₂)ₘaryl, —(C(R³)₂)ₚOR⁴, —(C(R³)₂)ₚNR⁵R⁶, —(C(R³)₂)ₘheteroaryl, —(C(R³)₂)ₚC(O)OR⁴, —(C(R³)₂)ₚN₃, —(C(R³)₂)ₚS(O)₂R⁷, —(C(R³)₂)ₚC(O)R⁷, —(C(R³)₂)ₚS(O)₂N(R⁶)₂, —(C(R³)₂)ₚC(O)N(R⁴)₂, benzo-fused heterocycloalkyl or benzo-fused cycloalkyl, wherein each said aryl and each said heteroaryl of R¹ is optionally independently substituted with Z;
R² is H, alkyl, —(C(R³)₂)ₘaryl, —(C(R³)₂)ₚOR⁴ or —(C(R³)₂)ₚNR⁵R⁶;
A is —CH₂— or —C(O)—;
each Y is independently selected from the group consisting of halogen, CN, —OR⁴, alkyl, —C(O)N(R⁶)₂, —O-haloalkyl, —NR⁵R⁶, -alkyleneC(O)N(R⁶)₂, —C(O)Oalkyl, -alkyleneOR⁶, —S(O)₂R⁷, —C(O)R⁷, -alkyleneS(O)₂N(R⁶)₂, —S(O)₂N(R⁶)₂, cycloalkyl, heterocycloalkyl, haloalkyl, aryl, heteroaryl, —SR⁷, —O-Q-L-R⁹, —O-Q-S(O)₂N(R⁶)₂, —O-Q-C(O)N(R⁶)₂, —O-Q-N(R⁶)C(O)N(R⁶)₂;
each Q is a divalent radical independently selected from alkylene-, -alkenylene-, -alkynylene-, -cycloalkylene-, -heterocycloalkylene-, and -alkylene-cycloalkylene-;
each L is independently selected from —O—, —S—, —S(O)—, —S(O)₂—, —C(O)—, and —OC(O)—;
each n is independently 0 to 5;
each m is independently 0 to 5;
p is 1 to 5;
each R³ is independently selected from the group consisting of H, alkyl, cycloalkyl and —OR⁶;
each R⁴ is independently selected from the group consisting of H, alkyl, aryl, cycloalkyl, heteroaryl and alkyleneOR⁶, wherein each said aryl and each said heteroaryl of R⁴ is optionally independently substituted with Z;
each R⁵ is independently selected from H, alkyl, heteroalkyl, aryl, heteroaryl, heterocycloalkyl, —S(O)₂R⁷, —C(O)R⁷, —C(O)N(R⁶)₂, S(O)₂N(R⁶)₂, —C(O)N(R⁶)₂, and —C(O)OR⁷;
each R⁶ is independently selected from the group consisting of H, alkyl, heteroalkyl, aryl, cycloalkyl, heterocycloalkyl, and heteroaryl, wherein each said aryl and each said heteroaryl of R⁶ is optionally independently substituted with Z;
each R⁷ is alkyl, aryl, heteroalkyl, heteroaryl, cycloalkyl, haloalkyl, aralkyl, hydroxyalkyl, alkoxyalkyl, alkylene-N(R⁸)₂, heteroaralkyl or heterocycloalkyl, wherein each said aryl and each said heteroaryl of R⁷ is optionally independently substituted with Z;
each R⁸ is independently selected from the group consisting of H, alkyl, aryl, cycloalkyl and heteroaryl;
each Z is independently selected from halogen, alkyl, —OR⁶, —CN, -haloalkyl, —C(O)N(R⁶)₂, —NR⁵R⁶, -cycloalkyl, -alkyleneOR⁶, -alkyleneNR⁵R⁶, and -alkyleneC(O)N(R⁶)₂; and
each R⁹ is independently selected from H, alkyl, cycloalkyl, heterocycloalkyl, aryl, and heteroaryl, wherein each said aryl and each said heteroaryl of R⁹ is optionally independently substituted with Z.

In another embodiment, the present invention provides a compound of the formula:

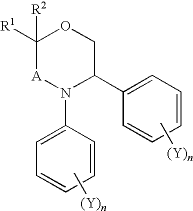

or a pharmaceutically acceptable salt, solvate, ester or prodrug thereof, wherein:
R¹ is alkyl, —(C(R³)₂)ₘaryl, —(C(R³)₂)ₚOR⁴, —(C(R³)₂)ₚNR⁵R⁶, —(C(R³)₂)ₘheteroaryl, —(C(R³)₂)ₚC(O)OR⁴, —(C(R³)₂)ₚN₃, —(C(R³)₂)ₚS(O)₂R⁷, —(C(R³)₂)ₚC(O)R⁷, —(C(R³)₂)ₚS(O)₂N(R⁶)₂, —(C(R³)₂)ₚC(O)N(R⁴)₂, benzo-fused heterocycloalkyl or benzo-fused cycloalkyl;
R² is H, alkyl, —(C(R³)₂)ₘaryl, —(C(R³)₂)ₚOR⁴ or —(C(R³)₂)ₚNR⁵R⁶;
A is —CH₂— or —C(O)—;
Y is independently selected from the group consisting of halogen, CN, —OR⁴, alkyl, —C(O)N(R⁶)₂, —O-haloalkyl, —NR⁵R⁶, -alkyleneC(O)N(R⁶)₂, —C(O)Oalkyl, -alkyleneOR⁶, —S(O)₂R⁷, —C(O)R⁷, -alkyleneS(O)₂N(R⁶)₂, —S(O)₂N(R⁶)₂, cycloalkyl, heterocycloalkyl, haloalkyl, aryl, heteroaryl and —SR⁷;
each m is independently 0 to 5;
n is 0 to 5;
p is 1 to 5;
R³ is independently selected from the group consisting of H, alkyl, cycloalkyl and —OR⁶;
R⁴ is independently selected from the group consisting of H, alkyl, aryl, heteroaryl and alkyleneOR⁶;
R⁵ is H, alkyl, aryl, heteroaryl, heterocycloalkyl, —S(O)₂R⁷, —C(O)R⁷, —C(O)N(R⁶)₂ or —S(O)₂N(R⁶)₂;
R⁶ is independently selected from the group consisting of H, alkyl, aryl, cycloalkyl and heteroaryl;
R⁷ is alkyl, aryl, heteroalkyl, heteroaryl, cycloalkyl, haloalkyl, aralkyl, hydroxyalkyl, alkoxyalkyl, alkyleneN(R⁸)₂, heteroaralkyl or heterocycloalkyl; and
R⁸ is independently selected from the group consisting of H, alkyl, aryl, cycloalkyl and heteroaryl.

The compounds of Formula (I) are CB1 receptor antagonists and as such are useful for treating various conditions responsive to CB1 receptor antagonists. Such conditions include, but are not limited to: metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering or reducing waist circumference, treatment of dyslipidemia, insulin sensitivity, insulin resistance, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome and inflammatory bowel diseases. These and other embodiments are described in more detail below.

### DETAILED DESCRIPTION OF THE INVENTION

In one embodiment, the present invention provides a compound, or a pharmaceutically acceptable salt, solvate, ester, or prodrug thereof, of Formula (I) as described above.

In another embodiment, in Formula (I), is a compound of Formula (I-A):

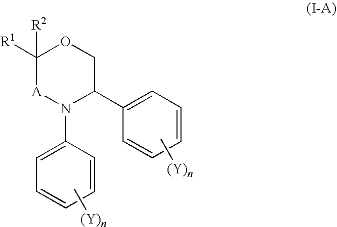

or a pharmaceutically acceptable salt, solvate, ester or prodrug thereof wherein R¹, R², A, each Y, and each n is selected independently and as defined in Formula (I).

In one embodiment, in Formula (I), is a compound of Formula (I-B):

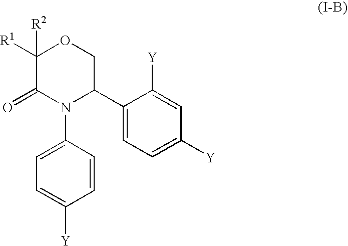

or a pharmaceutically acceptable salt, solvate, ester or prodrug thereof wherein R¹, R², and each Y is selected independently and as defined in Formula (I).

In another embodiment, in Formula (I), is a compound of Formula (I-C):

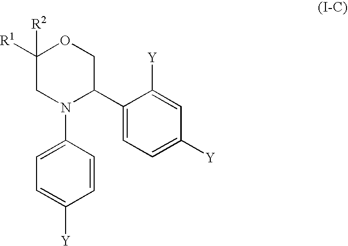

or a pharmaceutically acceptable salt, solvate, ester or prodrug thereof wherein R¹, R², and each Y is selected independently and as defined in Formula (I).

As used herein, the phrase, “compound(s) of the invention,” refers to a morpholine (or morpholinone) compound (or compounds) encompassed by Formula (I), Formula (I-A), Formula (I-B), and/or Formula (I-C), and/or pharmaceutically acceptable salts, solvates, esters, and prodrugs thereof.

In another embodiment, the present invention provides a pharmaceutical composition comprising a therapeutically effective amount of at least one compound of the invention, or a pharmaceutically acceptable salt, solvate, ester or prodrug thereof, and at least one pharmaceutically acceptable carrier.

In another embodiment, the present invention provides a pharmaceutical composition comprising a therapeutically effective amount of at least one compound of the invention, or a pharmaceutically acceptable salt, solvate, ester or prodrug thereof, together with one or more additional active agents suitable for treating a disease or condition responsive to CB1 receptor antagonism, optionally together with at least one pharmaceutically acceptable carrier.

In another embodiment, the present invention provides a method of treating a disease or disorder responsive to CB1 receptor antagonism in a patient. Non-limiting examples of such diseases or disorders include metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering of waist circumference, treatment of dyslipidemia, insulin sensitivity, insulin resistance, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome and inflammatory bowel diseases. Each of the listed methods comprises administering to the patient an effective amount of at least one compound of the invention, or a pharmaceutically acceptable salt, solvate, ester or prodrug thereof, alone or optionally together with one or more additional active agents suitable for use in treating the disease(s) or disorder(s) with which said patient is afflicted. Non-limiting examples of such additional active agents are described hereinbelow.

In another embodiment, in Formula (I) and/or Formula (I-A), A is —CH₂—.

In another embodiment, in Formula (I) and/or Formula (I-A), A is —C(O)—.

In another embodiment, in Formula (I), Formula (I-A), Formula (I-B), and/or Formula (I-C), R² is H.

In another embodiment, in Formula (I), Formula (I-A), Formula (I-B), and/or Formula (I-C), each Y is independently selected from halogen.

In another embodiment, in Formula (I), Formula (I-A), Formula (I-B), and/or Formula (I-C), each Y is Cl.

In another embodiment, in Formula (I-B) or (I-C), each Y is chlorine and R² is H.

In another embodiment, in Formula (I) and/or Formula (I-A), each n is independently 1 or 2.

In another embodiment, in Formula (I), Formula (I-A), Formula (I-B), and/or Formula (I-C), m is 0.

In another embodiment, in Formula (I), Formula (I-A), Formula (I-B), and/or Formula (I-C), m is 1.

In another embodiment, in Formula (I), Formula (I-A), Formula (I-B), and/or Formula (I-C), m is 2.

In another embodiment, in Formula (I), Formula (I-A), Formula (I-B), and/or Formula (I-C), m is 3.

In another embodiment, in Formula (I), Formula (I-A), Formula (I-B), and/or Formula (I-C), p is 1.

In another embodiment, in Formula (I), Formula (I-A), Formula (I-B), and/or Formula (I-C), p is 2.

In another embodiment, in Formula (I), Formula (I-A), Formula (I-B), and/or Formula (I-C), p is 3.

In another embodiment, in Formula (I), Formula (I-A), Formula (I-B), and/or Formula (I-C), R³ is H.

In another embodiment, in Formula (I), Formula (I-A), Formula (I-B), and/or Formula (I-C), R¹ is alkyl, —(C(R³)₂)₂aryl, —(C(R³)₂)₂OR⁴, —(C(R³)₂)₂NR⁵R⁶, —(C(R³)₂)₂heteroaryl, —(C(R³)₂)₂C(O)OR⁴ or —(C(R³)₂)₂C(O)N(R⁴)₂.

In another embodiment, in Formula (I), Formula (I-A), Formula (I-B), and/or Formula (I-C), R² is H, alkyl, —(C(R³)₂)₂aryl, —(C(R³)₂)₂OR⁴, —(C(R³)₂)₂OR⁴ or —(C(R³)₂)₂NR⁵R⁶.

In another embodiment, in Formula (I), Formula (I-A), Formula (I-B), and/or Formula (I-C), R¹ is —(CH₂)-aryl wherein said aryl is substituted with 1 to 5 halogens, —(CH₂)₂C(O)OR⁴, —(CH₂)₂OR⁴ or —(CH₂)₂NR⁵R⁶; or alternatively R¹ is

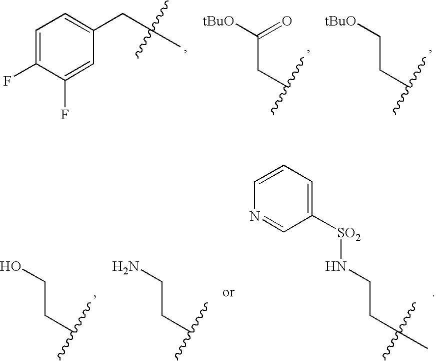

In another embodiment, in Formula (I), Formula (I-A), Formula (I-B), and/or Formula (I-C), R⁴ is hydrogen or alkyl.

In another embodiment, in Formula (I), Formula (I-A), Formula (I-B), and/or Formula (I-C), R⁵ is hydrogen.

In another embodiment, in Formula (I), Formula (I-A), Formula (I-B), and/or Formula (I-C), R⁶ is hydrogen.

In another embodiment, in Formula (I), Formula (I-A), Formula (I-B), and/or Formula (I-C), R⁵ is —SO₂alkyl, —SO₂aryl, —SO₂heteroaryl or —SO₂cycloalkyl; or alternatively R⁵ is —SO₂aryl or —SO₂heteroaryl.

In another embodiment, in Formula (I), Formula (I-A), Formula (I-B), and/or Formula (I-C), R¹ is —(CH₂)₂C(O)OR⁴ or —(CH₂)₂OR⁴.

Non-limiting examples of compounds of the invention include:

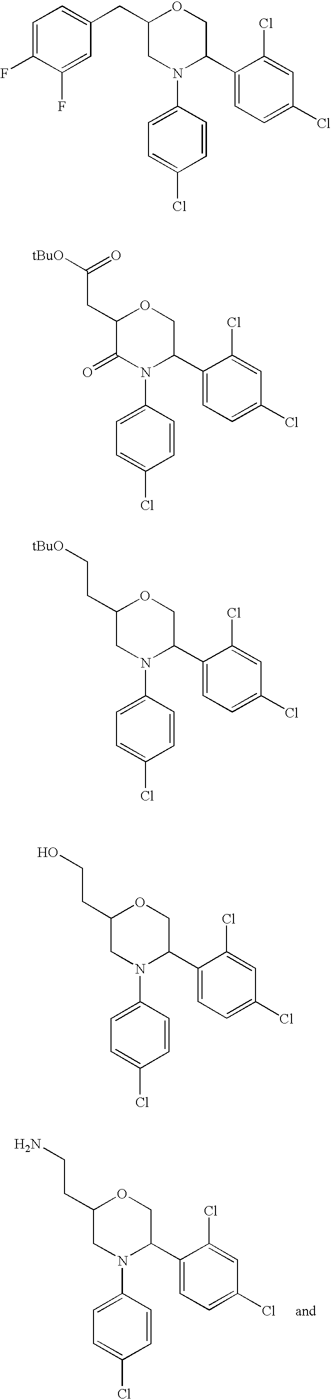

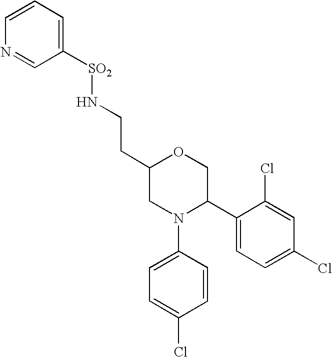

or a pharmaceutically acceptable salt, solvate, ester or prodrug thereof.

One of ordinary skill will recognize that the compounds shown above have stereogenic centers. Thus, the compounds shown above include all possible stereoisomers.

In the various embodiments described herein, R¹ is alkyl, —(C(R³)₂)ₘaryl, —(C(R³)₂)ₚOR⁴, —(C(R³)₂)ₚNR⁵R⁶, —(C(R³)₂)ₘheteroaryl, —(C(R³)₂)ₚC(O)OR⁴, —(C(R³)₂)ₚN₃, —(C(R³)₂)ₚS(O)₂R⁷, —(C(R³)₂)ₚC(O)R⁷, —(C(R³)₂)ₚS(O)₂N(R⁶)₂ or —(C(R³)₂)ₚC(O)N(R⁴)₂, wherein each said aryl and each said heteroaryl of R¹ is optionally substituted with a group Z. Non-limiting examples of R¹ when R¹ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R¹ when R¹ is —(C(R³)₂)ₘaryl include, for example, -aryl, —CH₂-aryl, —CH₂CH₂-aryl, —CH(CH₃)-aryl, —C(CH₃)₂-aryl, —CH₂CH₂CH₂-aryl, —CH(CH₃)CH₂-aryl, —CH₂CH(CH₃)-aryl, —CH(CH₃)—(CH₂)₂-aryl, —(CH₂)₂—CH(CH₃)-aryl, —CH(cyclopropyl)-CH₂-aryl, —CH₂—CH(cyclopropyl)-aryl, —CH(phenyl)-aryl, etc, where “aryl” includes, for example, phenyl, naphthyl, etc., and wherein m and R³ are defined as above. Non-limiting examples of R¹ when R¹ is —(C(R³)₂)ₚOR⁴ include, for example, —CH₂—OR⁴, —CH₂CH₂—OR⁴, —CH(CH₃)—OR⁴, —C(CH₃)₂—OR⁴, —CH₂CH₂CH₂—OR⁴, —CH(CH₃)CH₂—OR⁴, —CH₂CH(CH₃)—OR⁴, —CH(CH₃)—(CH₂)₂—OR⁴, —(CH₂)₂—CH(CH₃)—OR⁴, —CH(cyclopropyl)-CH₂—OR⁴, —CH₂—CH(cyclopropyl)-OR⁴, —CH(phenyl)-OR⁴, and wherein p and R⁴ are defined as above. Non-limiting examples of R¹ when R¹ is —(C(R³)₂)ₚNR⁵R⁶ include for example where R³, R⁵, R⁶, and —(C(R³)₂)ₚ— are defined as above. Non-limiting examples of R¹ when R¹ is —(C(R³)₂)ₘheteroaryl include for example where R³ and —(C(R³)₂)ₘ— are defined as above, and “heteroaryl”-pyridyl, -azaindolyl, -benzimidazolyl, -benzofuranyl, -furanyl, -indolyl, etc. Non-limiting examples of R¹ when R¹ is —(C(R³)₂)ₚC(O)OR⁴ include for example where R³, R⁴ and —(C(R³)₂)ₚ— are defined as above. Non-limiting examples of R¹ when R¹ is —(C(R³)₂)ₚN₃ include for example where R³ and —(C(R³)₂)ₚ— are defined as above. Non-limiting examples of R¹ when R¹ is —(C(R³)₂)ₚS(O)₂R⁷, include for example where R⁷ and —(C(R³)₂)ₚ— are defined as above. Non-limiting examples of R¹ when R¹ is —(C(R³)₂)ₚC(O)R⁷ include for example where R⁷ and —(C(R³)₂)ₚ— are defined as above. Non-limiting examples of R¹ when R¹ is —(C(R³)₂)ₚS(O)₂N(R⁶)₂ include for example where R³, R⁶ and —(C(R³)₂)ₚ— are defined as above. Non-limiting examples of R¹ when R¹ is —(C(R³)₂)ₚC(O)N(R⁴)₂ include for example where R³, R⁴ and

—(C(R³)₂)ₚ— are as defined as above. Non-limiting examples of R¹ when R¹ is benzo-fused heterocycloalkyl includes 3,4-dihydro-2H-benzo[1,4]oxazinyl, chromanyl, 2,3-dihydro-1H-indolyl, 2,3-dihydro-1H-isoindolyl, 2,3-dihydro-benzofuranyl, 1,3-dihydro-isobenzofuranyl, 2,3-dihydro-benzo[b]thiophenyl, 1,3-dihydro-benzo[c]thiophenyl, etc. Non-limiting examples of R¹ when R¹ is benzo-fused cycloalkyl include 1,2,3,4-tetrahydronaphthyl, indanyl, bicyclo[4.2.0]octa-1,3,5-trienyl, etc.

In the various embodiments described herein, R² is H, alkyl, —(C(R³)₂)ₘaryl, —(C(R³)₂)ₚOR⁴ or —(C(R³)₂)ₚNR⁵R⁶. Non-limiting examples of R² when R² is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R² when R² is —(C(R³)₂)ₘaryl include, for example, -aryl, —CH₂-aryl, —CH₂CH₂-aryl, —CH(CH₃)-aryl, —C(CH₃)₂-aryl, —CH₂CH₂CH₂-aryl, —CH(CH₃)CH₂-aryl, —CH₂CH(CH₃)-aryl, —CH(CH₃)—(CH₂)₂-aryl, —(CH₂)₂—CH(CH₃)-aryl, —CH(cyclopropyl)-CH₂-aryl, —CH₂—CH(cyclopropyl)-aryl, —CH(phenyl)-aryl, etc, where “aryl” includes, for example, phenyl, naphthyl, etc., and wherein m and R³ are defined as above. Non-limiting examples of R² when R² is —(C(R³)₂)ₚOR⁴ include for example where R³, R⁴ and —(C(R³)₂)ₚ— are defined as above. Non-limiting examples of R² when R² is —(C(R³)₂)ₚNR⁵R⁶ include for example where R³, R⁵, R⁶, and —(C(R³)₂)ₚ— are defined as above.

In the various embodiments described herein, each Y is independently selected from the group consisting of halogen, CN, —OR⁴, alkyl, —C(O)N(R⁶)₂, —O-haloalkyl, —NR⁵R⁶, -alkyleneC(O)N(R⁶)₂, —C(O)Oalkyl, -alkyleneOR⁶, —S(O)₂R⁷, —C(O)R⁷, -alkyleneS(O)₂N(R⁶)₂, —S(O)₂N(R⁶)₂, cycloalkyl, heterocycloalkyl, haloalkyl, aryl, heteroaryl, —SR⁷, —O-Q-L-R⁹, —O-Q-S(O)₂N(R⁶)₂, —O-Q-C(O)N(R⁶)₂, —O-Q-N(R⁶)C(O)N(R⁶)₂, wherein each Q is a divalent radical independently selected from -alkylene-, -alkenylene-, -alkynylene-, -cycloalkylene-, -heterocycloalkylene-, and -alkylene-cycloalkylene-. When Y is —OR⁴, R⁴ is defined as described above. Non-limiting examples of Y when Y is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. When Y is —C(O)N(R⁶)₂, R⁶ is defined as described above. Non-limiting examples of Y when Y is —O-haloalkyl include —OCF₃, —OCHF₂, —OCH₂F, —OCH₂CF₃, —OCF₂CF₃, —OCH₂Br, —OCH₂Cl, —OCCl₃, etc. When Y is —NR⁵R⁶, R⁵ and R⁶ are defined as described above. Non-limiting examples of Y when Y is -alkyleneC(O)N(R⁶)₂ include —CH₂C(O)N(R⁶)₂, —CH(CH₃)C(O)N(R⁶)₂, —CH₂CH₂C(O)N(R⁶)₂, —CH₂CH₂CH₂C(O)N(R⁶)₂, —CH(CH₃)CH₂CH₂C(O)N(R⁶)₂, etc., wherein each R⁶ is defined as described herein. For example, the “—N(R⁶)₂” portion of -alkyleneC(O)N(R⁶)₂ of Y can be —NH₂, —N(CH₃)₂, —NH(CH₃), —NH(phenyl), —N(phenyl)₂, —NH(phenyl)CH₃, —NH(cyclopropyl), —NH(pyridyl), —NCH₃(phenyl), —NCH₃(pyridyl), —NCH₃(cyclopropyl), etc. Non-limiting examples of Y when Y is —C(O)Oalkyl, include —C(O)Omethyl, —C(O)Oethyl, —C(O)O(n-propyl), —C(O)O(iso-propyl), —C(O)O(n-butyl), —C(O)O(iso-butyl), —C(O)O(sec-butyl), —C(O)O(tert-butyl), —C(O)O(n-pentyl), —C(O)O(iso-pentyl), —C(O)O(neo-pentyl), —C(O)O(n-hexyl), —C(O)O(iso-hexyl), etc. Non-limiting examples of Y when Y is -alkyleneOR⁶ include —CH₂OR⁶, —CH(CH₃)OR⁶, —CH₂CH₂OR⁶, —CH₂CH₂CH₂OR⁶, —CH(CH₃)CH₂CH₂OR⁶, etc., wherein each R⁶ is defined as described herein. For example, the —OR⁶ portion of said -alkyleneOR⁶ of Y can be —OH, —OCH₃, —OCH₂CH₃, —OCH(CH₃)₂, —O-phenyl, —O-cyclopropyl, —O-pyridyl, etc. When Y is —S(O)₂R⁷, R⁷ is defined as described above. When Y is —C(O)R⁷, R⁷ is defined as described above. When Y is -alkyleneS(O)₂N(R⁶)₂, the alkylene portion thereof can include any of the alkylene groups described herein (e.g., —CH₂—, —CH₂CH₂—, —CH(CH₃)—, —CH₂CH₂—, —CH₂CH₂CH₂—, —CH(CH₃)CH₂CH₂—, etc.), while the “—N(R⁶)₂” portion of -alkyleneS(O)₂N(R⁶)₂ is defined as described herein above. When Y is —S(O)₂N(R⁶)₂ the “—N(R⁶)₂” portion of —S(O)₂N(R⁶)₂ is defined as described herein above. Non-limiting examples of Y when Y is cycloalkyl include cyclopropyl, cyclobutyl, cyclopentyl, cyclohexyl, adamantyl, norbornyl, etc. Non-limiting examples of Y when Y is heterocycloalkyl include morpholinyl, piperazinyl, piperidinyl, pyrrolidinyl, tetrahydrofuranyl, tetrahydrothiophenyl, tetrahydropyranyl, azetidinyl, etc. Non-limiting examples of Y when Y is haloalkyl include —CF₃, —CHF₂, —CH₂F, —CH₂CF₃, —CF₂CF₃, —CH₂Br, —CH₂Cl, —CCl₃, etc. Non-limiting examples of Y when Y is aryl include, for example, phenyl, naphthyl, pyridyl (e.g., 2-, 3-, and 4-pyridyl), quinolyl, etc. substituted with one or more (e.g., 1, 2, 3, or 4) Y¹ groups as defined herein. Non-limiting examples of Y when Y is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non-limiting examples of Y when Y is —SR⁷, R⁷ is defined as described above. Non-limiting examples of Q when Y is —O-Q-L-R⁹ (wherein L is defined above), —O-Q-S(O)₂N(R⁶)₂, —O-Q-C(O)N(R⁶)₂, and —O-Q-N(R⁶)C(O)N(R⁶)₂, are provided below.

In the various embodiments, described herein, each R³ is independently selected from the group consisting of H, alkyl, cycloalkyl and —OR⁶. Non-limiting examples of R³ when R³ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R³ when R³ is cycloalkyl include cyclopropyl, cyclobutyl, cyclopentyl, cyclohexyl, adamantyl, norbornyl, etc. When R³ is —OR⁶, the R⁶ portion of —OR⁶ is herein described as below and include the following non-limiting examples, H, —CH₃, -phenyl, -cyclopropyl, -pyridyl, -cyclopropyl, etc.

Each R⁴ is independently selected from the group consisting of H, alkyl, cycloalkyl, aryl, heteroaryl and -alkyleneOR⁶, wherein each said aryl and each said heteroaryl of R⁴ is optionally independently substituted with Z. Non-limiting examples of R⁴ when R⁴ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁴ when R⁴ is aryl include, for example, phenyl, naphthyl, pyridyl (e.g., 2-, 3-, and 4-pyridyl), quinolyl, etc. substituted with one or more (e.g., 1, 2, 3, or 4) Y¹ groups as defined herein. Non-limiting examples of R⁴ when R⁴ is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non limiting examples of R⁴ when R⁴ is -alkyleneOR⁶ include —CH₂OR⁶, —CH(CH₃)OR⁶, —CH₂CH₂OR⁶, —CH₂CH₂CH₂OR⁶, —CH(CH₃)CH₂CH₂OR⁶, etc., wherein each R⁶ is independently defined as described herein. For example, the —OR⁶ portion of said -alkyleneOR⁶ of R⁴ can be —OH, —OCH₃, —OCH₂CH₃, —OCH(CH₃)₂, —O-phenyl, —O-cyclopropyl, —O-pyridyl, etc.

Each R⁵ is independently selected from H, alkyl, aryl, heteroaryl, heterocycloalkyl, —S(O)₂R⁷, —C(O)R⁷, —C(O)N(R⁶)₂, —S(O)₂N(R⁶)₂, —C(O)N(R⁶)₂, and —C(O)OR⁷. Non-limiting examples of R⁵ when R⁵ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁵ when R⁵ is aryl include, for example, phenyl, naphthyl, pyridyl (e.g., 2-, 3-, and 4-pyridyl), quinolyl, etc. substituted with one or more (e.g., 1, 2, 3, or 4) Y¹ groups as defined herein. Non-limiting examples of R⁵ when R⁵ is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non-limiting examples of R⁵ when R⁵ is heterocycloalkyl include morpholinyl, piperazinyl, piperidinyl, pyrrolidinyl, tetrahydrofuranyl, tetrahydrothiophenyl, tetrahydropyranyl, azetidinyl, etc. When R⁵ is —S(O)₂R⁷, R⁷ is defined as described above. When R⁵ is —C(O)R⁷, R⁷ is defined as described above. When R⁵ is —C(O)N(R⁶)₂ or —S(O)₂N(R⁶)₂ each R⁶ is defined as described herein. For example, the “—N(R⁶)₂” portion of —C(O)N(R⁶)₂ of R⁵ can be —NH₂, —N(CH₃)₂, —NH(CH₃), —NH(phenyl), —N(phenyl)₂, —NH(phenyl)CH₃, —NH(cyclopropyl), —NH(pyridyl), —NCH₃(phenyl), —NCH₃(pyridyl), —NCH₃(cyclopropyl), etc.

Each R⁶ is independently selected from H, alkyl, heteroalkyl, aryl, cycloalkyl, heterocycloalkyl, and heteroaryl, wherein each said aryl and each said heteroaryl of R⁶ is optionally independently substituted with Z. Non-limiting examples of R⁶ when R⁶ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁶ when R⁶ is aryl include phenyl, naphthyl, etc., wherein said aryl may be unsubstituted or substituted with one or more Y¹ groups as defined herein. Non-limiting examples of R⁶ when R⁶ is cycloalkyl include cyclopropyl, cyclobutyl, cyclopentyl, cyclohexyl, adamantyl, norbornyl, etc. Non-limiting examples of R⁶ when R⁶ is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc.

Each R⁷ is independently selected from alkyl, aryl, heteroalkyl, heteroaryl, cycloalkyl, haloalkyl, aralkyl, heteroaralkyl and heterocycloalkyl, wherein each said aryl and each said heteroaryl of R⁶ is optionally independently substituted with Z. Non-limiting examples of R⁷ when R⁷ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁷ when R⁷ is aryl include phenyl, naphthyl, etc., wherein said aryl may be unsubstituted or substituted with one or more Y¹ groups as defined herein. Non-limiting examples of R⁷ when R⁷ is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non-limiting examples of R⁷ when R⁷ is cycloalkyl include cyclopropyl, cyclobutyl, cyclopentyl, cyclohexyl, adamantyl, norbornyl, etc. Non-limiting examples of R⁷ when R⁷ is haloalkyl include —CF₃, —CHF₂, —CH₂F, —CH₂CF₃, —CF₂CF₃, —CH₂Br, —CH₂Cl, —CCl₃, etc. Non-limiting examples of R⁷ when R⁷ is heterocycloalkyl include morpholinyl, piperazinyl, piperidinyl, pyrrolidinyl, tetrahydrofuranyl, tetrahydrothiophenyl, tetrahydropyranyl, azetidinyl, etc. Non-limiting examples of R⁷ when R⁷ is aralkyl include benzyl, 2-phenethyl and naphthalenylmethyl. Non-limiting examples of R⁷ when R⁷ is heteroaralkyl include pyridylmethyl, and quinolin-3-ylmethyl. Non-limiting examples of R⁷ when R⁷ is heterocycloalkyl is heterocycloalkyl include morpholinyl, piperazinyl, piperidinyl, pyrrolidinyl, tetrahydrofuranyl, tetrahydrothiophenyl, tetrahydropyranyl, azetidinyl, etc.

Each R⁸, each R⁹, and each Z is independently defined as described herein.

The compounds of the invention, or pharmaceutically acceptable salts, solvates, esters or prodrugs thereof, are preferably purified to a degree suitable for use as a pharmaceutically active substance. That is, the compounds of the invention can have a purity of 95 wt % or more (excluding adjuvants such as pharmaceutically acceptable carriers, solvents, etc., which are used in formulating the compounds of the invention into a conventional form, such as a pill, capsule, IV solution, etc. suitable for administration into a patient). In other embodiments, the purity can be 97 wt % or more, or 99 wt % or more. A purified compound of the invention includes a single isomer having a purity, as discussed above, of 95 wt % or more, 97 wt % or more, or 99 wt % or more, as discussed above. For example, purified compounds of the invention can exist in forms exhibiting purities of 95 wt % or more, 97 wt % or more, or 99 wt % or more.

Alternatively, purified compounds of the invention can include a mixture of isomers, where the amount of impurity (i.e., compounds or other contaminants, exclusive of adjuvants as discussed herein) is 5 wt % or less, 3 wt % or less, or 1 wt % or less. For example, purified compounds of the invention can be present in an isomeric mixture of compounds of Formula (I), where the ratio of the amounts of the two isomers is approximately 1:1, and the combined amount of the two isomers is 95 wt % or more, 97 wt % or more, or 99 wt % or more.

As used above, and throughout this disclosure, the following terms, unless otherwise indicated, shall be understood to have the following meanings:

“DCE” means dichloroethane.

“DIAD” means diisopropylazodicarboxylate.

“DMSO” means dimethylsulfoxide.

“DPPA” means diphenylphosphoryl azide.

“EDCI” means 1-(3-dimethylaminopropyl)-3-ethylcarbodiimide hydrochloride.

“Et” means ethyl.

“EtOAc” means ethyl acetate.

“EtOH” mean ethanol.

“HOBt” means 1-hydroxybenzotriazole.

“LDA” means lithium diisopropyl amide.

“LHMDS” means lithium bis(trimethyl silyl)-amide.

“Me” means methyl.

“MeOH” means methanol.

“MsCl” means mesyl chloride or methanesulfonyl chloride.

“Ms” means mesyl or methanesulfonyl.

“Mammal” means humans and other mammalian animals.

“Patient” includes both human and animals. In one embodiment, said patient is a human. In another embodiment, said patient is a non-human animal. In another embodiment, the patient is a companion animal. For purposes of the present invention, the term “companion” animal shall be understood to include housecats (feline), dogs (canine), rabbit species, horses (equine), guinea pigs, rodents (e.g., squirrels, rats, mice, gerbils, and hamsters), primates (e.g., monkeys) and avians, such as pigeons, doves, parrots, parakeets, macaws, canaries, and the like.

“PS-DIEA” means diisopropylethyl amine functionalized polystyrene.

“PS-isocyante” means isocyanate functionalized polystyrene.

“PS-trisamine” means trisamine functionalized polystyrene.

“RT” means room temperature.

“TFA” means trifluoroacetic acid.

“TFAA” means trifluoroacetic anhydride.

“THF” means tetrahydrofuran.

“DMF” means N,N-dimethylformamide

“Cbz” means benzyloxycarbonyl

“Boc” means tert-butoxycarbonyl

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl),

—NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkylene” or “alkylenyl” means a difunctional group obtained by removal of a hydrogen atom from an alkyl group that is defined above. Non-limiting examples of alkylene include methylene, ethylene and propylene. “Lower alkylene” means an alkylene having about 1 to 6 carbon atoms in the chain, which may be straight or branched.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkenylene” means a difunctional group obtained by removal of a hydrogen from an alkenyl group that is defined above. Non-limiting examples of alkenylene include —CH═CH—, —C(CH₃)═CH—, and —CH═CHCH₂—.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Alkynylene” means a difunctional group obtained by removal of a hydrogen from an alkynyl group that is defined above. Non-limiting examples of alkenylene include —C≡C— and —CH₂C≡C—.

“Aryl” means an aromatic monocyclic or multicyclic ring system comprising about 6 to about 14 carbon atoms, or about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, or about 5 to about 10 ring atoms, in which one or more of the ring atoms is an element other than carbon, for example nitrogen, oxygen or sulfur, alone or in combination. In some embodiments, heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl, indazolyl, and the like, in which there is at least one aromatic ring.

“Aralkyl”, “arylalkyl”, or “alkylene-aryl” means an aryl-alkyl- group in which the aryl and alkyl are as previously described. In some embodiments, aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. In some embodiments, alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Cycloalkyl” can also mean a cycloalkyl wherein a single moiety (e.g., carbonyl) can simultaneously replace two available hydrogens on the same carbon atom on a ring system. A non-limiting example of such moiety is:

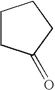

“Cycloalkylene” means a difunctional group obtained by removal of a hydrogen atom from a cycloalkyl group that is defined above. Non-limiting examples of cycloalkylene include

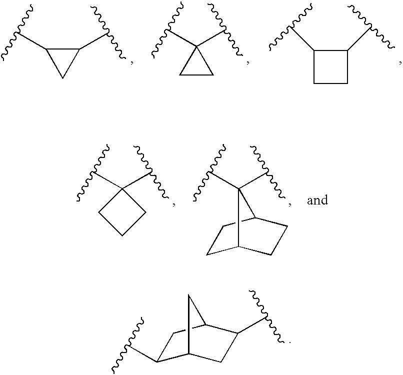

“Halogen” or “halo” means fluorine, chlorine, bromine, or iodine. In some embodiments, halogen is selected from fluorine, chlorine and bromine.

“Haloalkyl” means an alkyl as defined above wherein one or more hydrogen atoms on the alkyl are replaced by a halo group as defined above.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

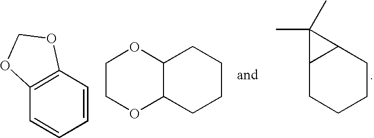

“Heterocyclyl” or “Heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” can also mean a heterocyclyl wherein a single moiety (e.g., carbonyl) can simultaneously replace two available hydrogens on the same carbon atom on a ring system. Example of such moiety is pyrrolidone:

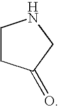

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

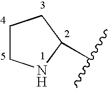

there is no —OH attached directly to carbons marked 2 and 5.

It should also be noted that in hetero-atom containing ring systems of this invention, both the heteroatom and carbon atom of said ring systems can be optionally substituted with a “ring system substituent”, where allowed by the appropriate valency rules.

It should also be noted that tautomeric forms of the compounds of The invention, including salts, solvates, esters, and prodrugs thereof are also contemplated herein. For example, the moieties:

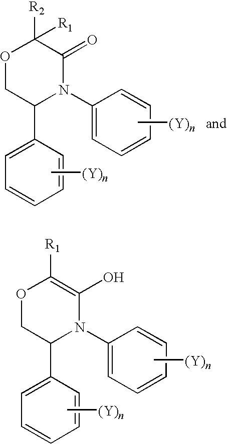

(e.g., when R² is H) are considered equivalent in such embodiments of this invention.

“Heterocyclylalkyl” or “heterocycloalkyl” means a heterocyclyl or heterocycloalkyl moiety as defined above linked via an alkyl moiety (defined above) to a parent core. Non-limiting examples of suitable heterocyclylalkyls or heterocycloalkyls include piperidinylmethyl, piperazinylmethyl and the like.

“Alkynylalkyl” means an alkynyl-alkyl- group in which the alkynyl and alkyl are as previously described. In some embodiments, alkynylalkyls contain a lower alkynyl and a lower alkyl group. The bond to the parent moiety is through the alkyl. Non-limiting examples of suitable alkynylalkyl groups include propargylmethyl.

“Heteroaralkyl”, “Heteroarylalkyl” or “alkylene-heteroaryl” means a heteroaryl-alkyl- group in which the heteroaryl and alkyl are as previously described. In some embodiments, heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. In some embodiments, hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. In some embodiments, acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

“Benzo-fused-cycloalkyl” or “Benzocycloalkyl” means a phenyl ring fused to a cycloalkyl, as defined above, so that each phenyl ring shares two ring carbon atoms with the cycloalkyl ring. The benzo-fused-cycloalkyl or benzocycloalkyl, can be optionally substituted with 1 to 3 “ring system substituents” as defined above. Non-limiting examples of benzo-fused cycloalkyls include the following:

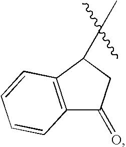

indanyl and tetradehydronaphthyl:

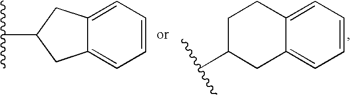

and non-limiting examples of a dibenzo-fused cycloalkyls are fluorenyl:

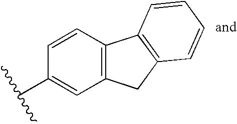

acenaphthenyl:

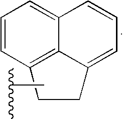

“Benzo-fused-heterocycloalkyl”, “benzo-fused-heterocyclyl” or “benzoheterocyclyl” means a phenyl ring fused to a heterocycloalkyl or heterocyclyl ring, as defined above, wherein said benzo-fused-heterocycloalkyl, benzo-fused-heterocyclyl or benzoheterocyclyl can be optionally substituted with 1 to 3 “ring system substituents” as defined above. Non-limiting examples of suitable benzo-fused-heterocycloalkyl, benzo-fused-heterocyclyl or benzoheterocyclyl groups include the following:

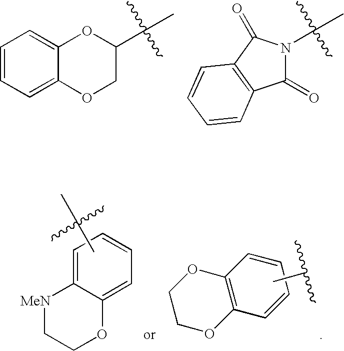

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

When used herein, the term “independently”, in reference to the substitution of a parent moiety with one or more substituents, means that the parent moiety may be substituted with any of the listed substituents, either individually or in combination, and any number of chemically possible substituents may be used. As a non-limiting example, a phenyl independently substituted with one or more alkyl or halo substituents can include, chlorophenyl, dichlorophenyl, trichlorophenyl, tolyl, xylyl, 2-chloro-3-methylphenyl, 2,3-dichloro-4-methylphenyl, etc.

As used herein, the term “composition” is intended to encompass a product comprising the specified ingredients in the specified amounts, as well as any product which results, directly or indirectly, from combination of the specified ingredients in the specified amounts.

The wavy line [symbol] , as a bond generally indicates a mixture of, or either of, the possible isomers, e.g., containing (R)- and (S)-stereochemistry. For example.

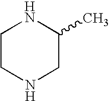

means containing both

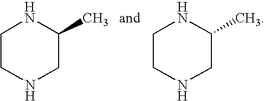

Moreover, when the stereochemistry of a chiral center (or stereogenic center) is not expressly indicated, a mixture of, or any of the individual possible isomers are contemplated. Thus, for example,

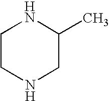

means containing

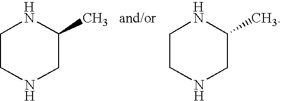

Lines drawn into the ring systems, such as, for example:

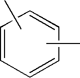

indicate that the indicated line (bond) may be attached to any of the substitutable ring atoms.

As well known in the art, a bond drawn from a particular atom wherein no moiety is depicted at the terminal end of the bond indicates a methyl group bound through that bond to the atom, unless stated otherwise. For example:

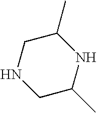

represents

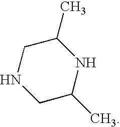

It should also be noted that any carbon or heteroatom with unsatisfied valences in the text, schemes, examples, structural formulae, and any Tables herein is assumed to have the hydrogen atom or atoms to satisfy the valences.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure”, it is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocyclyl, R², etc.) occurs more than one time in any constituent or in Formula (A), Formula (B), Formula (I) or other formulas describing the compounds of the invention, its definition on each occurrence is independent of its definition at every other occurrence.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of The invention or a pharmaceutically acceptable salt, solvate, ester or prodrug of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference thereto.

For example, if a compound of the invention or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of The invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of the invention incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93 (3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5 (1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of the invention can form salts which are also within the scope of this invention. Reference to a compound of the invention is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66 (1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of the invention, and salts, solvates, esters and prodrugs thereof, may exist in their tautomeric form (for example, as an amide or imino ether). All such tautomeric forms are contemplated herein as part of the present invention.

The compounds of the invention may contain asymmetric or chiral centers; and, therefore, exist in different stereoisomeric forms. It is intended that all stereoisomeric forms of the compounds of The invention as well as mixtures thereof, including racemic mixtures, form part of the present invention. In addition, the present invention embraces all geometric and positional isomers. For example, if a compound of The invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of The invention may be atropisomers (e.g., substituted biaryls) and are considered as part of this invention. Enantiomers can also be separated by use of chiral HPLC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of the invention incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of The invention can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of the invention, and of the salts, solvates, esters and prodrugs of the compounds of the invention, are intended to be included in the present invention.

In still another embodiment, the present invention provides a composition comprising at least one compound of the invention, or a pharmaceutically acceptable salt, solvate, ester or prodrug thereof, and a pharmaceutically acceptable carrier.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

As used herein, the term “pharmaceutical combination” means a combination of two or more pharmaceutically active compounds. Such combination can be in any form. The term “pharmaceutical combination” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units. A pharmaceutical combination can also include two or more pharmaceutical compounds administered separately, e.g., in two or more separate dosage units.

Unit dosage forms, without limitation, can include tablets, pills, capsules, sustained release pills, sustained release tablets, sustained release capsules, powders, granules, or in the form of solutions or mixtures (i.e., elixirs, tinctures, syrups, emulsions, suspensions). For example, one or more compounds of The invention, or salts or solvates thereof, may be combined, without limitation, with one or more pharmaceutically acceptable liquid carriers such as ethanol, glycerol, or water, and/or one or more solid binders such as, for example, starch, gelatin, natural sugars (e.g., glucose or β-lactose), and/or natural or synthetic gums (e.g., acacia, tragacanth, or sodium alginate), carboxymethylcellulose, polyethylene glycol, waxes and the like, and/or disintegrants, buffers, preservatives, anti-oxidants, lubricants, flavorings, thickeners, coloring agents, emulsifiers and the like. In addition, the unit dosage forms can include, without limitation, pharmaceutically acceptable lubricants (e.g., sodium oleate, sodium stearate, magnesium stearate, sodium benzoate, sodium acetate, and sodium chloride) and disintegrators (e.g., starch, methyl cellulose, agar, bentonite, and xanthan gum).

The amount of excipient or additive can range from about 0.1 to about 90 weight percent of the total weight of the treatment composition or therapeutic combination. One skilled in the art would understand that the amount of carrier(s), excipients and additives (if present) can vary.

The compounds of the invention, or pharmaceutically acceptable salts, solvates, esters or prodrugs thereof, can be administered in any suitable form, e.g., alone, or in combination with a pharmaceutically acceptable carrier, excipient or diluent in a pharmaceutical composition, according to standard pharmaceutical practice. The compounds of the invention, or pharmaceutically acceptable salts, solvates, esters or prodrugs thereof, can be administered orally or parenterally, including intravenous, intramuscular, interperitoneal, subcutaneous, rectal, or topical routes of administration.

Pharmaceutical compositions comprising at least one compound of the invention, or a pharmaceutically acceptable salt, solvate, ester or prodrug thereof can be in a form suitable for oral administration, e.g., as tablets, troches, capsules, lozenges, aqueous or oily suspensions, dispersible powders or granules, emulsions, syrups, or elixirs. Oral compositions may be prepared by any conventional pharmaceutical method, and may also contain sweetening agents, flavoring agents, coloring agents, and preserving agents.

The amount of compound of the invention, or a pharmaceutically acceptable salt, solvate, ester or prodrug thereof, administered to a patient can be determined by a physician based on the age, weight, and response of the patient, as well as by the severity of the condition treated. For example, the amount of compound of the invention, or a pharmaceutically acceptable salt, solvate, ester or prodrug thereof, administered to the patient can range from about 0.1 mg/kg body weight per day to about 60 mg/kg/d. In some embodiments, the dose is about 0.5 mg/kg/d to about 40 mg/kg/d.

In another embodiment, the present invention provides a method of treating, reducing, or ameliorating a disease or condition selected from the group consisting of metabolic syndrome, obesity, waist circumference, lipid profile, insulin sensitivity, insulin resistance, neuroinflammatory disorders, cognitive disorders, psychosis, addictive behavior, gastrointestinal disorders, and cardiovascular conditions, in a patient in need thereof, comprising administering to said patient an effective amount of at least one compound of The invention, or a pharmaceutically acceptable salt, solvate, ester or prodrug thereof.

In yet another embodiment, the present invention provides a method of treating, reducing, or ameliorating obesity, in a patient in need thereof, comprising administering to said patient an effective amount of at least one compound of The invention, or a pharmaceutically acceptable salt, solvate, ester or prodrug thereof.

In yet another embodiment, the present invention provides a method of treating, reducing, or ameliorating metabolic syndrome, obesity, waist circumference, lipid profile, insulin sensitivity, insulin resistance, neuroinflammatory disorders, cognitive disorders, psychosis, addictive behavior, gastrointestinal disorders, and cardiovascular conditions, in a patient in need thereof, comprising administering to said patient an effective amount of a composition comprising at least one compound of The invention, or a pharmaceutically acceptable salt, solvate, ester or prodrug thereof and a pharmaceutically acceptable carrier.

In yet another embodiment, the present invention provides a method of treating, reducing, or ameliorating obesity, in a patient in need thereof, comprising administering to said patient an effective amount of a composition comprising at least one compound of The invention, or a pharmaceutically acceptable salt, solvate, ester or prodrug thereof and a pharmaceutically acceptable carrier.

The compounds of the invention can be useful as CB1 receptor antagonists for treating, reducing, or ameliorating metabolic syndrome, obesity, waist circumference, lipid profile, insulin sensitivity, insulin resistance, neuroinflammatory disorders, cognitive disorders, psychosis, addictive behavior (e.g., smoking cessation), gastrointestinal disorders, and cardiovascular conditions (e.g., elevated cholesterol and triglyceride levels). It is contemplated that the compounds of The invention of the present invention, or pharmaceutically acceptable salts, solvates, esters or prodrugs thereof, can be useful in treating one or more the conditions or diseases listed above. In particular, the compounds of The invention of the present invention are useful in treating obesity.

The selective CB1 receptor antagonist compound of the invention, or a pharmaceutically acceptable salt, solvate, ester or prodrug thereof, can be administered in a therapeutically effective amount and manner to treat the specified condition. The daily dose of the selective CB1 receptor antagonist of The invention (or pharmaceutically acceptable salts, solvates, esters or prodrugs thereof) administered to a mammalian patient or subject can range from about 1 mg/kg to about 50 mg/kg (where the units mg/kg refer to the amount of selective CB1 receptor antagonist compound of The invention per kg body weight of the patient), or about 1 mg/kg to about 25 mg/kg, or about 1 mg/kg to about 10 mg/kg.

Alternatively, the daily dose can range from about 1 mg to about 50 mg, or about 1 mg to about 25 mg, or about 5 mg to about 20 mg. Although a single administration of the selective CB1 receptor antagonist compound of the invention, or salts, solvates, or esters thereof, can be efficacious, multiple dosages can also be administered. The exact dose, however, can readily be determined by the attending clinician and will depend on such factors as the potency of the compound administered, the age, weight, condition and response of the patient.

The treatment compositions of the present invention can be administered in any conventional dosage form, preferably an oral dosage form such as a capsule, tablet, powder, cachet, suspension or solution. The formulations and pharmaceutical compositions can be prepared using conventional pharmaceutically acceptable and conventional techniques.

The compounds of the invention may also be used in conjunction with an additional therapeutic agent or agents for the treatment of the diseases, conditions and/or disorders described herein. Thus, in another embodiment, methods of treatment that include administering compounds of the present invention in combination with other therapeutic agents are also provided.

Suitable other therapeutic agents that may be used in combination with compounds of the invention include anti-obesity agents such as apolipoprotein-B secretion/microsomal triglyceride transfer protein (apo-B/MTP) inhibitors, 11β.-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (e.g., sibutramine), sympathomimetic agents, β3 adrenergic receptor agonists, dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y antagonists (e.g., NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345; 6,326,375, and 6,566,367; U.S. Publication Nos. 2002/0151456, 2003/036652, 2004/192705, 2003/036652, 2004/072847, and 2005/033048; and PCT Publication No. WO 03/082190), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine.TM. available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists and the like. Other anti-obesity agents are well known or would be readily apparent to one of ordinary skill in the art.

In one embodiment, compounds of the invention are combined with anti-obesity agents selected from the group consisting of orlistat, sibutramine, bromocriptine, ephedrine, leptin, pseudoephedrine, PYY₃₋₃₆ or an analog thereof, and 2-oxo-N-(5-phenylpyrazinyl)spiro-[isobenzofuran-1(3H), 4′-piperidine]-1′-carboxamide.

Representative anti-obesity agents for use in the combinations, pharmaceutical compositions, and methods of the present invention can be prepared using methods known in the art, for example, sibutramine can be prepared as described in U.S. Pat. No. 4,929,629; bromocriptine can be prepared as described in U.S. Pat. No. 3,752,814 and U.S. Pat. No. 3,752,888; orlistat can be prepared as described in U.S. Pat. No. 5,274,143; U.S. Pat. No. 5,420,305; U.S. Pat. No. 5,540,917; and U.S. Pat. No. 5,643,874; PYY₃₋₃₆ (including analogs) can be prepared as described in U.S. Publication No. 2002/0141985 and WO 03/027637; and the NPY Y5 receptor antagonist 2-oxo-N-(5-phenyl-pyrazinyl)spiro[isobenzofuran-1(3H), 4′-piperidine]-1′-carboxamide can be prepared as described in U.S. Publication No. 2002/0151456. Other useful NPY Y5 receptor antagonists include those described in PCT Publication No. 03/082190, such as 3-oxo-N-(5-phenyl-2-pyrazinyl)-spiro[isobenzofuran-1(3H), 4′-piperidine]-1′-carboxamide; 3-oxo-N-(7-trifluoromethylpyrido[3,2-b]pyridin-2-yl)-spiro-[isobenzofuran-1(3H), 4′-piperidine]-1′-carboxamide; N-[5-(3-fluorophenyl)-2-pyrimidinyl]-3-oxospiro-isobenzofuran-1(3H), [4′-piperidine]-1′-carboxamide; trans-3′-oxo-N-(5-phenyl-2-pyrimidinyl)spiro[cyclohexane-1,1′(3′H)-isobenzofuran]-4-carboxamide; trans-3′-oxo-N-[1-(3-quinolyl)-4-imidazolyl]spiro[cyclohexane-1,1′(3′H)-isobenzofuran]-4-carboxamide; trans-3-oxo-N-(5-phenyl-2-pyrazinyl)spiro[4-azaisobenzofuran-1(3H), 1′-cyclohexane]-4′-carboxamide, trans-N-[5-(3-fluorophenyl)-2-pyrimidinyl]-3-oxospiro[5-azaisobenzofuran-1(3H), 1′-cyclohexane]-4′-carboxamide; trans-N-[5-(2-fluorophenyl)-2-pyrimidinyl]-3-oxospiro[5-azaisobenzofuran-1(3H), 1′-cyclohexane]-4′-carboxamide; trans-N-[1-(3,5-difluorophenyl)-4-imidazolyl]-3-oxospiro[7-azaisobenzofuran-1(3H), 1′-cyclohexane]-4′-carboxamide; trans-3-oxo-N-(1-phenyl-4-pyrazolyl)spiro[4-azaisobenzofuran-1(3H), 1′-cyclohexane]-4′-carboxamide; trans-N-[1-(2-fluorophenyl)-3-pyrazolyl]-3-oxospiro[6-azaisobenzofuran-1(3H), 1′-cyclohexane]-4′-carboxamide; trans-3-oxo-N-(I-phenyl-3-pyrazolyl)spiro[6-azaisobenzofuran-1(3H), 1′-cyclohexane]-4′-carboxamide; trans-3-oxo-N-(2-phenyl-1,2,3-triazol-4-yl)spiro[6-azaisobenzofuran-1(3H), 1′-cyclohexane]-4′-carboxamide; and pharmaceutically acceptable salts, solvates, esters or prodrugs thereof. All of the above recited patents and publications are incorporated herein by reference.

Other suitable therapeutic agents that may be administered in combination with one or more compounds of the invention include therapeutic agents designed to treat tobacco abuse (e.g., nicotine receptor partial agonists, bupropion hypochloride (also known under the tradename Zyban™) and nicotine replacement therapies), agents to treat erectile dysfunction (e.g., dopaminergic agents, such as apomorphine), ADD/ADHD agents (e.g., Ritalin™, Strattera™, Concerta™ and Adderall™), and agents to treat alcoholism, such as opioid antagonists (e.g., naltrexone (also known under the tradename ReVia™) and nalmefene), disulfuram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™).

Other therapeutic agents that may administered in combination with one or more compounds of the invention include antihypertensive agents, anti-inflammatory agents (e.g., COX-2 inhibitors), antidepressants (e.g., fluoxetine hydrochloride (Prozac™)), cognitive improvement agents (e.g., donepezil hydrochloride (Aircept™) and other acetylcholinesterase inhibitors), neuroprotective agents (e.g., memantine), antipsychotic medications (e.g., ziprasidone (Geodon™), risperidone (Risperdal™), asenapine (e.g., in a fast-dissolving sublingual tablet or other form), and olanzapine (Zyprexa™)), insulin and insulin analogs (e.g., LysPro insulin), GLP-1 (7-37) (insulinotropin) and GLP-1 (7-36)-NH₂, sulfonylureas and analogs thereof (e.g., chlorpropamide, glibenclamide, tolbutamide, tolazamide, acetohexamide, Glypizide™, glimepiride, repaglinide, meglitinide; biguanides: metformin, phenformin, buformin), α2-antagonists and imidazolines (e.g., midaglizole, isaglidole, deriglidole, idazoxan, efaroxan, fluparoxan), other insulin secretagogues (e.g., linogliride, A-4166), glitazones (e.g., ciglitazone, Actose™, pioglitazone, englitazone, troglitazone, darglitazone, Avandia™, BRL49653), fatty acid oxidation inhibitors (e.g., clomoxir, etomoxir), α-glucosidase inhibitors (e.g., acarbose, miglitol, emiglitate, voglibose, MDL-25,637, camiglibose, MDL-73,945), β-agonists (e.g., BRL 35135, BRL 37344, RO 16-8714, ICI D7114, CL 316,243), phosphodiesterase inhibitors (e.g., L-386,398), lipid-lowering agents (e.g., benfluorex, fenfluramine), vanadate and vanadium complexes (e.g., Naglivan™) and peroxovanadium complexes, amylin antagonists, glucagon antagonists, gluconeogenesis inhibitors, somatostatin analogs, antilipolytic agents (e.g., nicotinic acid, acipimox, WAG 994, pramlintide (Symlin™), AC 2993, nateglinide, aldose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol lowering compounds.

Non-limiting examples of cholesterol lowering compounds suitable for administration in combination with one or more compounds of the invention include cholesterol biosynthesis inhibitors, cholesterol absorption inhibitors, HMG-CoA reductase inhibitors, HMG-COA synthase inhibitors, HMG-CoA reductase or synthase gene expression inhibitors, CETP inhibitors, bile acid sequesterants, fibrates, ACAT inhibitors, squalene synthetase inhibitors, squalene epoxidase inhibitors, sterol biosynthesis inhibitors, nicotinic acid derivatives, bile acid sequestrants, inorganic cholesterol sequestrants, AcylCoA:Cholesterol O-acyltransferase inhibitors, cholesteryl ester transfer protein inhibitors, fish oils containing Omega 3 fatty acids, natural water soluble fibers, plant stanols and/or fatty acid esters of plant stanols, low-density lipoprotein receptor activators, anti-oxidants and niacin.

A non-limiting list of cholesterol lowering compounds suitable for administration with one or more compounds of the invention include HMG CoA reductase inhibitor compounds such as lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin, fluvastatin, cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride); sterol biosynthesis inhibitors such as DMP-565; nicotinic acid derivatives (e.g., compounds comprising a pyridine-3-carboxylate structure or a pyrazine-2-carboxylate structure, including acid forms, salts, esters, zwitterions and tautomers) such as niceritrol, nicofuranose and acipimox (5-methylpyrazine-2-carboxylic acid 4-oxide); clofibrate; gemfibrazol; bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof; inorganic cholesterol sequestrants such as bismuth salicylate plus montmorillonite clay, aluminum hydroxide and calcium carbonate antacids; ileal bile acid transport (“IBAT”) inhibitors (or apical sodium co-dependent bile acid transport (“ASBT”) inhibitors) such as benzothiepines, for example the therapeutic compounds comprising a 2,3,4,5-tetrahydro-1-benzothiepine 1,1-dioxide structure such as are disclosed in PCT Patent Application WO 00/38727 which is incorporated herein by reference; AcylCoA:Cholesterol O-acyltransferase (“ACAT”) Inhibitors such as avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60 (1); 55-93, which is incorporated by reference herein; Cholesteryl Ester Transfer Protein (“CETP”) Inhibitors such as those disclosed in PCT Patent Application No. WO 00/38721 and U.S. Pat. No. 6,147,090, which are incorporated herein by reference; probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE-402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13:1005-12, herein incorporated by reference; fish oils containing Omega 3 fatty acids (3-PUFA); natural water soluble fibers, such as psyllium, guar, oat and pectin; plant stanols and/or fatty acid esters of plant stanols, such as sitostanol ester used in BENECOL® margarine; and the substituted azetidinone or substituted β-lactam sterol absorption inhibitors.

Compounds that inhibit cholesterol absorption in the small intestine are well known in the art and are described, for example, in U.S. RE 37,721; U.S. Pat. No. 5,631,356; U.S. Pat. No. 5,767,115; U.S. Pat. No. 5,846,966; U.S. Pat. No. 5,698,548; U.S. Pat. No. 5,633,246; U.S. Pat. No. 5,656,624; U.S. Pat. No. 5,624,920; U.S. Pat. No. 5,688,787; U.S. Pat. No. 5,756,470; US Publication No. 2002/0137689; WO 02/066464; WO 95/08522 and WO96/19450.

As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a mammal or human. Particularly useful sterol absorption inhibitors include hydroxy-substituted azetidinone compounds and substituted β-lactam compounds, for example those disclosed in U.S. Pat. Nos. 5,767,115, 5,624,920, 5,668,990, 5,656,624 and 5,688,787, which are herein incorporated by reference in their entirety. These patents, respectively, disclose hydroxy-substituted azetidinone compounds and substituted β-lactam compounds useful for lowering cholesterol and/or in inhibiting the formation of cholesterol-containing lesions in mammalian arterial walls. U.S. Pat. No. 5,756,470, U.S. Patent Application No. 2002/0137690, U.S. Patent Application No. 2002/0137689 and PCT Patent Application No. WO 2002/066464 (each of which is herein incorporated by reference in its entirety) disclose sugar-substituted azetidinones and amino acid substituted azetidinones useful for preventing or treating atherosclerosis and reducing plasma cholesterol levels.

In one embodiment, substituted azetidinones useful in the compositions, therapeutic combinations and methods of the present invention are represented by Formula (II) below:

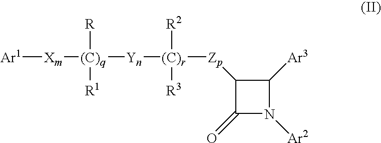

or pharmaceutically acceptable salts, solvates, or esters of the compounds of Formula (II), wherein, in Formula (II) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(lower alkyl)₂-;

R and R² are independently selected from the group consisting of —OR⁶, —OC(O)R⁶, —OC(O)OR⁹ and —OC(O)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀CONR⁶R⁷, -(lower alkylene)COOR⁶, —CH═CH—C(O)OR⁶, —CF₃, —CN, —NO₂ and halogen;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —SO₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

Certain compounds useful in the therapeutic compositions or combinations of the invention may have at least one asymmetrical carbon atom and therefore all isomers, including enantiomers, diastereomers, stereoisomers, rotamers, tautomers and racemates of the compounds of Formula II-XIII (where they exist) are contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of the Formulae II-XIII. Isomers may also include geometric isomers, e.g., when a double bond is present.

Those skilled in the art will appreciate that for some of the compounds of the Formulae II-XIII, one isomer may show greater pharmacological activity than other isomers.

Preferred compounds of Formula (II) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —OC(O)R⁶, —OC(O)OR⁹ and —OC(O)NR⁶R⁷, defined above).

The sum of m, n, p, q and r is preferably 2, 3 or 4, more preferably 3. Preferred are compounds OF Formula (II) wherein m, n and r are each zero, q is 1 and p is 2.

Also preferred are compounds of Formula (II) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (II) wherein p, q and n are each zero, r is 1, m is 2, X is —CH₂— and R² is —OR⁶, especially when R⁶ is hydrogen.

Another group of preferred compounds of Formula (II) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

### Substituted Azetidinones of Formula (III)

In a preferred embodiment, a substituted azetidinone of Formula (II) useful in the compositions, therapeutic combinations and methods of the present invention is represented by Formula (III) (ezetimibe) below:

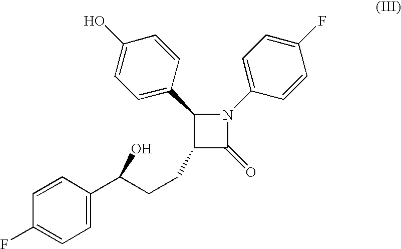

or pharmaceutically acceptable salts, solvates, or esters of the compound of Formula (III). The compound of Formula (III) can be in anhydrous or hydrated form. A product containing ezetimibe compound is commercially available as ZETIA® ezetimibe formulation from MSP Pharmaceuticals.

Compounds of Formula (II) can be prepared by a variety of methods well known to those skilled in the art, for example such as are disclosed in U.S. Pat. Nos. 5,631,365, 5,767,115, 5,846,966, 6,207,822, 6,627,757, 6,093,812, 5,306,817, 5,561,227, 5,688,785, and 5,688,787, each of which is incorporated herein by reference.

### Substituted Azetidinones of Formula (IV)

Alternative substituted azetidinones useful in the compositions, therapeutic combinations and methods of the present invention are represented by Formula (IV) below:

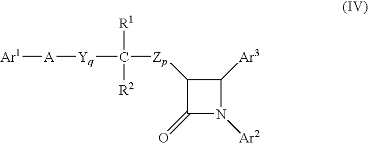

or a pharmaceutically acceptable salt thereof or a solvate thereof, or an ester thereof, wherein, in Formula (IV) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(lower alkyl)₂-;

A is selected from —O—, —S—, —S(O)— or —S(O)₂—;

R¹ is selected from the group consisting of —OR⁶, —OC(O)R⁶, —OC(O)OR⁹ and —OC(O)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —OC(O)R⁶, —OC(O)OR⁹, —O(CH₂)₁₋₅OR⁹, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂-lower alkyl, —NR⁶S(O)₂-aryl, —C(O)NR⁶R⁷, —COR⁶, —SO₂NR⁶R⁷, S(O)₀₋₂-alkyl, S(O)₀₋₂-aryl, —O(CH₂)₁₋₁₀—C(O)OR⁶, —O(CH₂)₁₋₁₀C(O)NR⁶R⁷, o-halogeno, m-halogeno, o-lower alkyl, m-lower alkyl, -(lower alkylene)-C(O)OR⁶, and —CH═CH—C(O)OR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Methods for making compounds of Formula (IV) are well known to those skilled in the art. Non-limiting examples of suitable methods are disclosed in U.S. Pat. No. 5,688,990, which is incorporated herein by reference.

### Substituted Azetidinones of Formula (V)

In another embodiment, substituted azetidinones useful in the compositions, therapeutic combinations and methods of the present invention are represented by Formula (V):

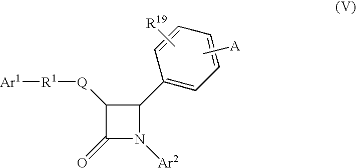

or a pharmaceutically acceptable salt thereof or a solvate thereof, or an ester thereof, wherein, in Formula (V) above:

A is selected from the group consisting of R²-substituted heterocycloalkyl, R²-substituted heteroaryl, R²-substituted benzo-fused heterocycloalkyl, and R²-substituted benzo-fused heteroaryl;

Ar¹ is aryl or R³-substituted aryl;

Ar² is aryl or R⁴-substituted aryl;

Q is a bond or, with the 3-position ring carbon of the azetidinone, forms the spiro group

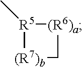

and

R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

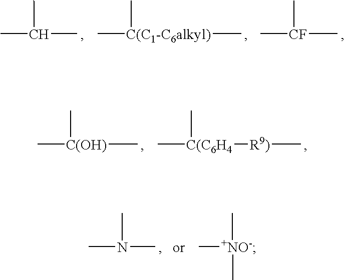

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;

and when Q is a bond, R¹ also can be selected from:

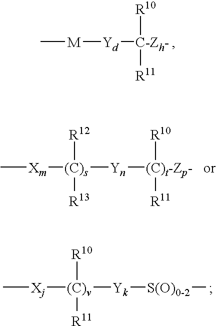

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —OC(O)R¹⁴, —OC(O)OR¹⁶ and —OC(O)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —C(O)R¹⁴, hydroxy(C₁-C₆)alkyl, (C₁-C₆)alkoxy(C₁-C₆)alkyl, NO₂, —S(O)₀₋₂R¹⁶, —S(O)₂NR¹⁴R¹⁵ and —(C₁-C₆ alkylene)C(O)OR¹⁴; when R² is a substituent on a heterocycloalkyl ring, R² is as defined, or R² is ═O or

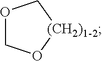

and, where R² is a substituent on a substitutable ring nitrogen, R² is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

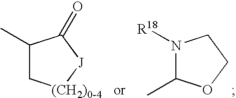

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —OC(O)R¹⁴, —OC(O)OR¹⁶, —O(CH₂)₁₋₅OR¹⁴, —OC(O)NR¹⁴R¹⁵, —NR¹⁴R¹⁵, —NR¹⁴C(O)R¹⁵, —NR¹⁴C(O)OR¹⁶, —NR¹⁴C(O)NR¹⁵R¹⁹, —NR¹⁴S(O)₂R¹⁶, —C(O)OR¹⁴, —C(O)NR¹⁴R¹⁵, —C(O)R¹⁴, —S(O)₂NR¹⁴R¹⁵, S(O)₀₋₂R¹⁶, —O(CH₂)₁₋₁₀—C(O)OR¹⁴, —O(CH₂)₁₋₁₀C(O)NR¹⁴R¹⁵, —(C₁-C₆ alkylene)-C(O)OR¹⁴, —CH═CH—C(O)OR¹⁴, —CF₃, —CN, —NO₂ and halogen;

R⁸ is hydrogen, (C₁-C₆)alkyl, aryl (C₁-C₆)alkyl, —C(O)R¹⁴ or —C(O)OR¹⁴;

R⁹ and R¹⁷ are independently 1-3 groups independently selected from the group consisting of hydrogen, (C₁-C₆)alkyl, (C₁-C₆)alkoxy, —C(O)OH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

Methods for making compounds of Formula (V) are well known to those skilled in the art. Non-limiting examples of suitable methods are disclosed in U.S. Pat. No. 5,656,624, which is incorporated herein by reference.

### Substituted Azetidinones of Formula (VI)

In another embodiment, substituted azetidinones useful in the compositions, therapeutic combinations and methods of the present invention are represented by Formula (VI):

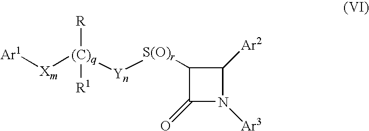

or a pharmaceutically acceptable salt thereof or a solvate thereof, or an ester thereof, wherein, in Formula (VI) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(lower alkyl)₂-;

R is —OR⁶, —OC(O)R⁶, —OC(O)OR⁹ or —OC(O)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅—OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, —CF₃, —CN, —NO₂, halogen, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, —S(O)₀₋₂R⁹, —O(CH₂)₁₋₁₀—C(O)OR⁶, —O(CH₂)₁₋₁₀C(O)NR⁶R⁷, —CF₃, —CN, —NO₂ and halogen.

Methods for making compounds of Formula (VI) are well known to those skilled in the art. Non-limiting examples of suitable methods are disclosed in U.S. Pat. No. 5,624,920, which is incorporated herein by reference.

### Substituted Azetidinones of Formula (VII)

In another embodiment, substituted azetidinones useful in the compositions, therapeutic combinations and methods of the present invention are represented by Formula (VII):

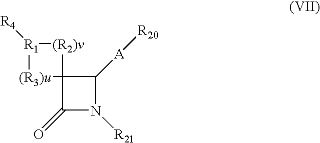

or a pharmaceutically acceptable salt thereof or a solvate thereof, or an ester thereof, wherein:

R¹ is:

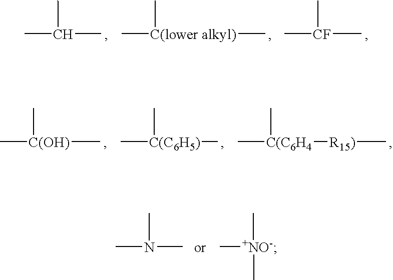

R² and R³ are independently selected from the group consisting of: —CH₂—, —CH(lower alkyl)-, —C(lower alkyl)₂-, —CH═CH— and —C(lower alkyl)=CH—; or R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)=CH—, v is 1; provided that when R³ is —CH═CH— or —C(lower alkyl)=CH—, u is 1; provided that when v is 2 or 3, each R² can be the same or different; and provided that when u is 2 or 3, each R³ can be the same or different;

R⁴ is selected from B—(CH₂)ₘC(O)—, wherein m is 0, 1, 2, 3, 4 or 5; B—(CH₂)_{q}—, wherein q is 0, 1, 2, 3, 4, 5 or 6; B—(CH₂)ₑ—Z—(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-; B—(C₄-C₆ alkadienylene)-; B—(CH₂)ₜ—Z—(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6;

B—(CH₂)ₐ—Z—(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is a C₃-C₆ cycloalkyl and s is 0, 1, 2, 3, 4, 5 or 6; or

R¹ and R⁴ together form the group number

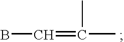

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

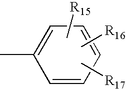

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹(O)₂SNH—, (R¹¹(O)₂S)₂N—, —S(O)₂NH₂, —S(O)₀₋₂R⁸, tert-butyldimethyl-silyloxymethyl, —C(O)R¹², —C(O)OR¹⁹, —C(O)N(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and

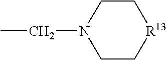

for substitution on ring carbon atoms, and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —C(O)OH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy,

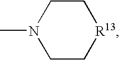

—N(R⁸)(R⁹), lower alkyl, phenyl or R⁷-phenyl;

R¹³ is selected from —O—, —CH₂—, —NH—, —N(lower alkyl)- or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzo-fused heteroaryl, W-substituted benzo-fused heteroaryl and cyclopropyl, wherein heteroaryl is as defined above.

Methods for making compounds of Formula (VII) are well known to those skilled in the art. Non-limiting examples of suitable methods are disclosed in U.S. Pat. No. 5,698,548, which is incorporated herein by reference.

### Substituted Azetidinones of Formula (VIII)

In another embodiment, substituted azetidinones useful in the compositions, therapeutic combinations and methods of the present invention are represented by Formulas (VIIIA) and (VIIIB):

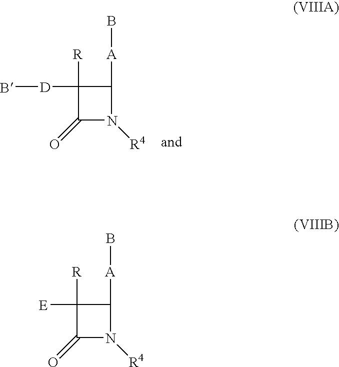

or a pharmaceutically acceptable salt, solvate, or ester thereof,
wherein:

A is —CH═CH—, —C≡C— or —(CH₂)ₚ— wherein p is 0, 1 or 2;

B is

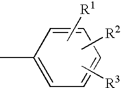

B′ is

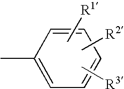

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R³, R^{1′}, R^{2′}, and R^{3′} are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶(O)₂SNH— and —S(O)₂NH₂;

R⁴ is

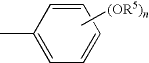

wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt, solvate, or ester thereof.

### Sterol Absorption Inhibitors of Formula (IX)

In another embodiment, sterol absorption inhibitors useful in the compositions and methods of the present invention are represented by Formula (IX):

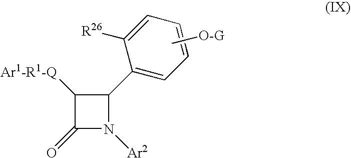

or a pharmaceutically acceptable salt, solvate, or ester thereof, wherein, in Formula (IX) above,

R²⁶ is H or OG¹;

G and G¹ are independently selected from the group consisting of

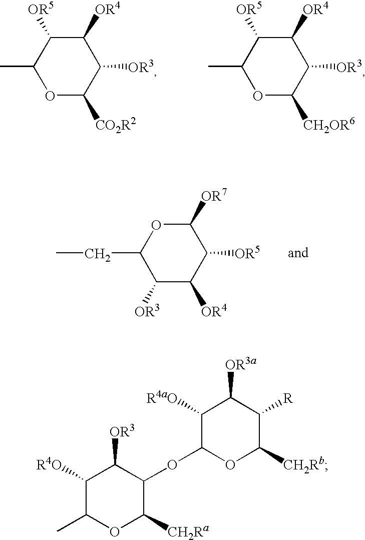

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

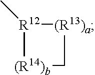

and

R¹ is selected from the group consisting of
—(CH₂)_{q}—, wherein q is 2-6, provided that when Q forms a spiro ring, q can also be zero or 1;
—(CH₂)ₑ-E-(CH₂)ᵣ, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)₉—, wherein V is C₃-C₆ cycloalkylene, f is 1-5 and g is 0-5, provided that the sum of f and g is 1-6;

R¹² is:

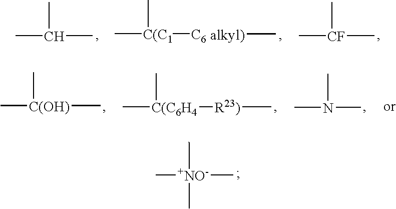

R¹³ and R¹⁴ are independently selected from the group consisting of

—CH₂—, —CH((C₁-C₆) alkyl)-, —C((C₁-C₆) alkyl)₂, —CH═CH— and —C((C₁-C₆) alkyl)=CH—; or

R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, each R¹³ can be the same or different; and

provided that when b is 2 or 3, each R¹⁴ can be the same or different;

and when Q is a bond, R¹ also can be:

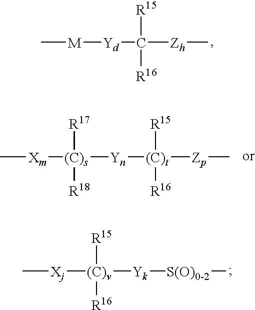

M is —O—, —S—, —S(O)— or —S(O)₂—;

X, Y and Z are independently selected from the group consisting of —CH₂—, —CH(C₁-C₆)alkyl- and —C((C₁-C₆)alkyl)₂;

R¹⁰ and R¹¹ are independently selected from the group consisting of 1-3 substituents independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR₁₉C(O)R²⁰, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halogen;

R¹⁵ and R¹⁷ are independently selected from the group consisting of —OR¹⁹, —OC(O)R¹⁹, —OC(O)OR²¹ and —OC(O)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is

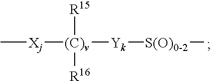

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are independently 1-3 groups independently selected from the group consisting of H, (C₁-C₆)alkyl, (C₁-C₆)alkoxy, —C(O)OH, NO₂, —NR¹⁹R²⁰, —OH and halogeno; and

R²⁵ is H, —OH or (C₁-C₆)alkoxy.

Methods for making compounds of Formula (IX) are well known to those skilled in the art. Non-limiting examples of suitable methods are disclosed in U.S. Pat. No. 5,756,470, which is incorporated herein by reference.

### Substituted Azetidinones of Formula (X)

In another embodiment, substituted azetidinones useful in the compositions and methods of the present invention are represented by Formula (X) below:

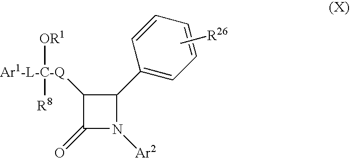

or a pharmaceutically acceptable salt, solvate, or ester thereof, wherein in Formula (X):

R¹ is selected from the group consisting of H, G, G¹, G², —SO₃H and —PO₃H;

G is selected from the group consisting of: H,

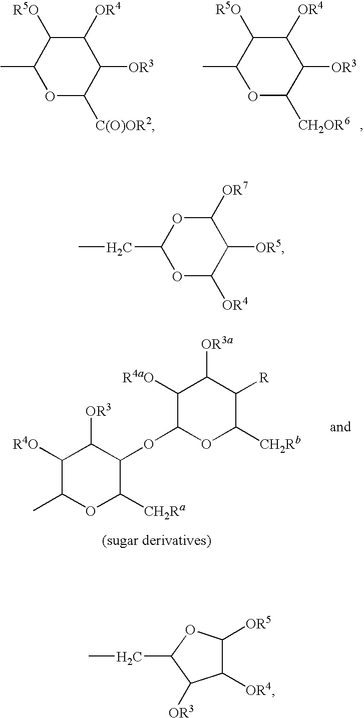

wherein R, R^{a} and R^{b} are each independently selected from the group consisting of H, —OH, halo, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N(C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

G¹ is represented by the structure:

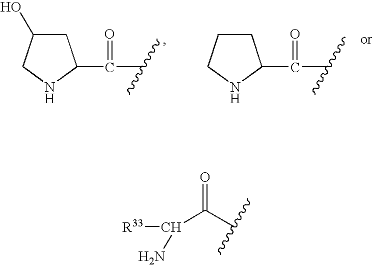

wherein R³³ is independently selected from the group consisting of unsubstituted alkyl, R³⁴-substituted alkyl, (R³⁵)(R³⁶)alkyl-,

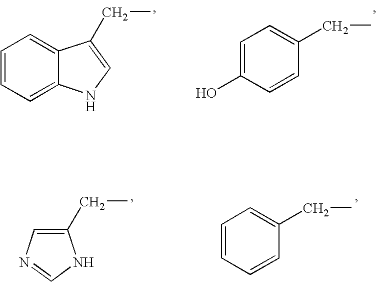

R³⁴ is one to three substituents, each R³⁴ being independently selected from the group consisting of HO(O)C—, HO—, HS—, (CH₃)S—, H₂N—, (NH₂)(NH)C(NH)—, (NH₂)C(O)— and HO(O)CCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure:

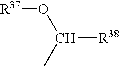

wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c) —OCH₃;
d) fluorine;
e) chlorine;
f) —O-G;
g) —O-G¹;
h) —O-G²;
i) —SO₃H; and
j) —PO₃H;
   provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:
a) a covalent bond;
b) —(CH₂)_{q}—, wherein q is 1-6;
c) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
d) —(C₂-C₆)alkenylene-;
e) —(CH₂)_{f}—V—(CH₂)_{g}—, wherein V is C₃-C₆cycloalkylene, f is 1-5 and g is 0-5, provided that the sum of f and g is 1-6; and
f)

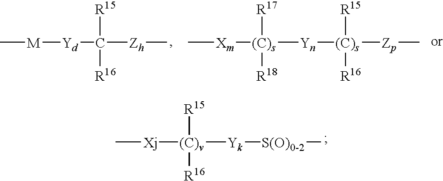

wherein M is —O—, —S—, —S(O)— or —S(O)₂—;

X, Y and Z are each independently selected from the group consisting of —CH₂—, —CH(C₁-C₆)alkyl- and —C((C₁-C₆)alkyl)₂-;

R⁸ is selected from the group consisting of H and alkyl;

R¹⁰ and R¹¹ are each independently selected from the group consisting of 1-3 substituents which are each independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR¹⁹C(O)R²⁹, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halo;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl; or

R¹⁵ and R¹⁶ together are ═O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-5, provided that the sum of j, k and v is 1-5;

Q is a bond, —(CH₂)_{q}—, wherein q is 1-6, or, with the 3-position ring carbon of the azetidinone, forms the spiro group

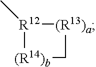

wherein R¹² is

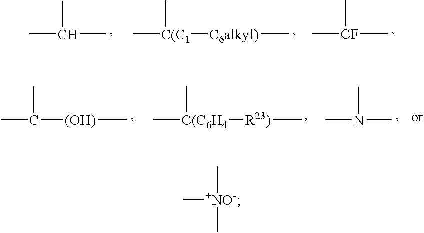

R¹³ and R¹⁴ are each independently selected from the group consisting of —CH₂—, —CH(C₁-C₆ alkyl)-, —C((C₁-C₆) alkyl)₂, —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, each R¹³ can be the same or different; and provided that when b is 2 or 3, each R¹⁴ can be the same or different;

and when Q is a bond and L is

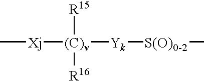

then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are each independently selected from the group consisting of 1-3 substituents which are each independently selected from the group consisting of H, (C₁-C₆)alkyl, (C₁-C₆)alkoxy, —C(O)OH, NO₂, —NR¹⁹R²⁰, —OH and halo; and

R²⁵ is H, —OH or (C₁-C₆)alkoxy.

Examples of compounds of Formula (X) which are useful in the methods and combinations of the present invention and methods for making such compounds are disclosed in U.S. patent application Ser. No. 10/166,942, filed Jun. 11, 2002, incorporated herein by reference.

### Substituted Azetidinones of Formulae (XI)-(XIII)

An example of a useful substituted azetidinone is one represented by the Formula (XI):

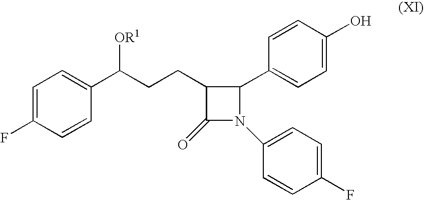

wherein R¹ is defined as above.

A more preferred compound is one represented by Formula (XII):

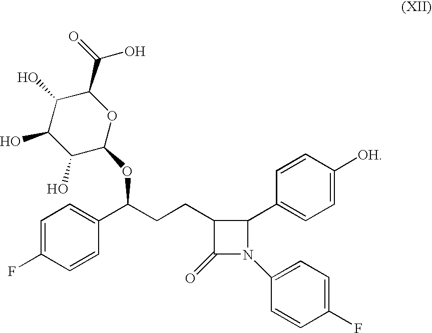

Another useful compound is represented by Formula (XIII):

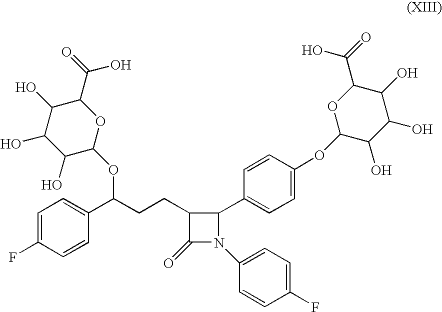

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, diphenyl azetidinones and derivatives disclosed in U.S. Patent Publication Nos. 2002/0039774, 2002/0128252, 2002/0128253 and 2002/0137689, 2004/063929, WO 2002/066464, U.S. Pat. Nos. 6,498,156 and 6,703,386, each of which is incorporated by reference herein.

Other sterol absorption inhibitors useful in the compositions, therapeutic combinations and methods of the present invention are described in WO 2004/005247, WO 2004/000803, WO 2004/000804, WO 2004/000805, WO 0250027, U.S. published application 2002/0137689, and the compounds described in L. Kværnø et al., Angew. Chem. Int. Ed., 2004, vol. 43, pp. 4653-4656, all of which are incorporated herein by reference. An illustrative compound of Kværnø et al. is:

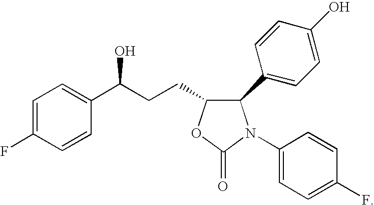

The compounds of Formulae II-XIII can be prepared by known methods, including the methods discussed above and, for example, in WO 93/02048, U.S. Pat. Nos. 5,306,817 and 5,561,227, herein incorporated by reference, which describe the preparation of compounds wherein —R¹-Q- is alkylene, alkenylene or alkylene interrupted by a hetero atom, phenylene or cycloalkylene; WO 94/17038 and U.S. Pat. No. 5,698,548, herein incorporated by reference, describe the preparation of compounds wherein Q is a spirocyclic group; WO 95/08532, U.S. Pat. No. 5,631,365, U.S. Pat. No. 5,767,115, U.S. Pat. No. 5,846,966, and U.S. R.E. 37,721, herein incorporated by reference, describe the preparation of compounds wherein —R¹-Q- is a hydroxy-substituted alkylene group; PCT/US95/03196, herein incorporated by reference, describes compounds wherein —R¹-Q- is a hydroxy-substituted alkylene attached to the Ar¹ moiety through an —O— or S(O)₀₋₂— group; and U.S. Ser. No. 08/463,619, filed Jun. 5, 1995, herein incorporated by reference, describes the preparation of compounds wherein —R¹-Q- is a hydroxy-substituted alkylene group attached to the azetidinone ring by a —S(O)₀₋₂— group. Each of the above patents or publications are herein incorporated by reference in their entirety.

The daily dose of the sterol absorption inhibitor(s) administered to the subject can range from about 0.1 to about 1000 mg per day, preferably about 0.25 to about 50 mg/day, and more preferably about 10 mg per day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

In another embodiment of the present invention, the compositions or therapeutic combinations described above comprise one or more selective CB1 receptor antagonist compounds of The invention in combination with one or more cholesterol biosynthesis inhibitors and/or lipid-lowering compounds discussed below.

Generally, a total daily dosage of cholesterol biosynthesis inhibitor(s) can range from about 0.1 to about 160 mg per day, and preferably about 0.2 to about 80 mg/day in single or 2-3 divided doses.

In another alternative embodiment, the compositions, therapeutic combinations or methods of the present invention can comprise at least one compound of The invention, or pharmaceutically acceptable salts, solvates, esters or prodrugs thereof, and one or more bile acid sequestrants (insoluble anion exchange resins), co-administered with or in combination with the compound of The invention, or a pharmaceutically acceptable salt, solvate, ester or prodrug thereof, and a substituted azetidinone or a substituted 3-lactam discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors that bind LDL from plasma to further reduce cholesterol levels in the blood.

Generally, a total daily dosage of bile acid sequestrant(s) can range from about 1 to about 50 grams per day, and preferably about 2 to about 16 grams per day in single or 2-4 divided doses.

In an alternative embodiment, the compositions or treatments of the present invention can comprise at least one compound of the invention, or pharmaceutically acceptable salts, solvates, esters or prodrugs thereof, and one or more IBAT inhibitors. The IBAT inhibitors can inhibit bile acid transport to reduce LDL cholesterol levels. Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions or treatments of the present invention can comprise at least one compound of the invention, or pharmaceutically acceptable salts, solvates, esters or prodrugs thereof, and nicotinic acid (niacin) and/or derivatives thereof. Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions or treatments of the present invention can comprise at least one compound of the invention, or pharmaceutically acceptable salts, solvates, esters or prodrugs thereof, and one or more AcylCoA:Cholesterol O-acyltransferase (“ACAT”) Inhibitors, which can reduce LDL and VLDL levels. ACAT is an enzyme responsible for esterifying excess intracellular cholesterol and may reduce the synthesis of VLDL, which is a product of cholesterol esterification, and overproduction of apo B-100-containing lipoproteins. Generally, a total daily dosage of ACAT inhibitor(s) can range from about 0.1 to about 1000 mg/day in single or 2-4 divided doses.

In another alternative embodiment, the compositions or treatments of the present invention can comprise at least one compound of the invention, or pharmaceutically acceptable salts, solvates, esters or prodrugs thereof, and one or more Cholesteryl Ester Transfer Protein (“CETP”) Inhibitors. CETP is responsible for the exchange or transfer of cholesteryl ester carrying HDL and triglycerides in VLDL. Pancreatic cholesteryl ester hydrolase (pCEH) inhibitors such as WAY-121898 also can be co-administered with or in combination.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions or treatments of the present invention can comprise at least one compound of the invention, or pharmaceutically acceptable salts, solvates, esters or prodrugs thereof, and probucol or derivatives thereof, which can reduce LDL levels.

Generally, a total daily dosage of probucol or derivatives thereof can range from about 10 to about 2000 mg/day, and preferably about 500 to about 1500 mg/day in single or 2-4 divided doses.

In another alternative embodiment, the compositions or treatments of the present invention can comprise at least one compound of the invention, or pharmaceutically acceptable salts, solvates, esters or prodrugs thereof, and low-density lipoprotein (LDL) receptor activators.

Generally, a total daily dosage of LDL receptor activator(s) can range from about 1 to about 1000 mg/day in single or 2-4 divided doses.

In another alternative embodiment, the compositions or treatments of the present invention can comprise at least one compound of the invention, or pharmaceutically acceptable salts, solvates, esters or prodrugs thereof, and fish oil. Generally, a total daily dosage of fish oil or Omega 3 fatty acids can range from about 1 to about 30 grams per day in single or 2-4 divided doses.

In another alternative embodiment, the compositions or treatments of the present invention can further comprise at least one compound of the invention, or pharmaceutically acceptable salts, solvates, esters or prodrugs thereof, and natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, the compositions or treatments of the present invention can comprise at least one compound of the invention, or pharmaceutically acceptable salts, solvates, esters or prodrugs thereof, and plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

In another alternative embodiment, the compositions or treatments of the present invention can comprise at least one compound of the invention, or pharmaceutically acceptable salts, solvates, esters or prodrugs thereof, and antioxidants, such as probucol, tocopherol, ascorbic acid, β-carotene and selenium, or vitamins such as vitamin B₆ or vitamin B₁₂. Generally, a total daily dosage of antioxidants or vitamins can range from about 0.05 to about 10 grams per day in single or 2-4 divided doses.

In another alternative embodiment, the compositions or treatments of the present invention can comprise at least one compound of the invention, or pharmaceutically acceptable salts, solvates, esters or prodrugs thereof, and monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E. Generally, a total daily dosage of these agents can range from about 0.01 to about 1000 mg/day in single or 2-4 divided doses.

Also useful with the present invention are compositions or therapeutic combinations that further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename Estratest.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17α-dihydroequilin sulfate, sodium 17α-estradiol sulfate, sodium 17β-dihydroequilin sulfate, sodium 17α-dihydroequilenin sulfate, sodium 17β-dihydroequilenin sulfate, sodium equilenin sulfate and sodium 17β-estradiol sulfate; available from Duramed Pharmaceuticals, Inc., Cincinnati, Ohio, under the tradename Cenestin;

(b) ethinyl estradiol (19-nor-17α-pregna-1,3,5(10)-trien-20-yne-3,17-diol; available by Schering Plough Corporation, Kenilworth, N.J., under the tradename Estinyl;

(c) esterified estrogen combinations such as sodium estrone sulfate and sodium equilin sulfate; available from Solvay under the tradename Estratab and from Monarch Pharmaceuticals, Bristol, Tenn., under the tradename Menest;

(d) estropipate (piperazine estra-1,3,5(10)-trien-17-one, 3-(sulfooxy)-estrone sulfate); available from Pharmacia & Upjohn, Peapack, N.J., under the tradename Ogen and from Women First Health Care, Inc., San Diego, Calif., under the tradename Ortho-Est; and

(e) conjugated estrogens (17α-dihydroequilin, 17α-estradiol, and 17β-dihydroequilin); available from Wyeth-Ayerst Pharmaceuticals, Philadelphia, Pa., under the tradename Premarin.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1,3,5(10)-triene-3,17β-diol hemihydrate) and norethindrone (17β-acetoxy-19-nor-17α-pregn-4-en-20-yn-3-one); which is available from Pharmacia & Upjohn, Peapack, N.J., under the tradename Activella;

(b) the combination of levonorgestrel (d(−)-13β-ethyl-17α-ethinyl-17β-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordette, and from Wyeth-Ayerst under the tradename Triphasil;

(c) the combination of ethynodiol diacetate (19-nor-17α-pregn-4-en-20-yne-3β, 17-diol diacetate) and ethinyl estradiol; available from G.D. Searle & Co., Chicago, Ill., under the tradename Demulen and from Watson under the tradename Zovia;

(d) the combination of desogestrel (13-ethyl-11-methylene-18,19-dinor-17α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept;

(e) the combination of norethindrone and ethinyl estradiol; available from Parke-Davis, Morris Plains, N.J., under the tradenames Estrostep and FemHRT, from Watson under the tradenames Microgestin, Necon, and Tri-Norinyl, from Ortho-McNeil under the tradenames Modicon and Ortho-Novum, and from Warner Chilcott Laboratories, Rockaway, N.J., under the tradename Ovcon;

(f) the combination of norgestrel ((±)-13-ethyl-17-hydroxy-18,19-dinor-17α-preg-4-en-20-yn-3-one) and ethinyl estradiol; available from Wyeth-Ayerst under the tradenames Ovral and Lo/Ovral, and from Watson under the tradenames Ogestrel and Low-Ogestrel;

(g) the combination of norethindrone, ethinyl estradiol, and mestranol (3-methoxy-19-nor-17α-pregna-1,3,5(10)-trien-20-yn-17-ol); available from Watson under the tradenames Brevicon and Norinyl;

(h) the combination of 17β-estradiol (estra-1,3,5(10)-triene-3,17β-diol) and micronized norgestimate (17α-17-(Acetyloxyl)-13-ethyl-18,19-dinorpregn-4-en-20-yn-3-one3-oxime); available from Ortho-McNeil under the tradename Ortho-Prefest;

(i) the combination of norgestimate (18,19-dinor-17-pregn-4-en-20-yn-3-one, 17-(acetyloxy)-13-ethyl-, oxime, (17(α)-(+)-) and ethinyl estradiol; available from Ortho-McNeil under the tradenames Ortho Cyclen and Ortho Tri-Cyclen; and

(j) the combination of conjugated estrogens (sodium estrone sulfate and sodium equilin sulfate) and medroxyprogesterone acetate (20-dione, 17-(acetyloxy)-6-methyl-, (6(α))-pregn-4-ene-3); available from Wyeth-Ayerst under the tradenames Premphase and Prempro.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3,20-dione); available from Solvay under the tradename Prometrium; and medroxyprogesterone acetate; available from Pharmacia & Upjohn under the tradename Provera.

In another alternative embodiment, the compositions, therapeutic combinations or methods of the present invention can comprise at least one compound of The invention, or pharmaceutically acceptable salts, solvates, esters or prodrugs thereof, and one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); alpha-blocking agents; kainite or AMPA receptor antagonists; leptin-lipolysis stimulated receptors; phosphodiesterase enzyme inhibitors; compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations and methods of the present invention can comprise at least one compound of the invention, or pharmaceutically acceptable salts, solvates, esters or prodrugs thereof, and one or more blood modifiers which are chemically different from the substituted azetidinone and substituted β-lactam compounds (such as compounds II-XIII above) and the lipid modulating agents discussed above, for example, they contain one or more different atoms, have a different arrangement of atoms or a different number of one or more atoms than the sterol absorption inhibitor(s) or lipid modulating agents discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor VIIa inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl}amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl]propylamides, substituted n-[(aminomethyl)phenyl]propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can comprise at least one compound of the invention, or pharmaceutically acceptable salts, solvates, esters or prodrugs thereof, and one or more cardiovascular agents which are chemically different from the substituted azetidinone and substituted β-lactam compounds (such as compounds II-XIII above) and the lipid modulating agents discussed above, for example, they contain one or more different atoms, have a different arrangement of atoms or a different number of one or more atoms than the sterol absorption inhibitor(s) or PPAR receptor activators discussed above. Useful cardiovascular agents include but are not limited to calcium channel blockers (clentiazem maleate, amlodipine besylate, isradipine, nimodipine, felodipine, nilvadipine, nifedipine, teludipine hydrochloride, diltiazem hydrochloride, belfosdil, verapamil hydrochloride, fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril, delapril hydrochloride, fosinopril sodium, libenzapril, moexipril hydrochloride, pentopril, perindopril, quinapril hydrochloride, quinaprilat, ramipril, spirapril hydrochloride, spiraprilat, teprotide, enalapril maleate, lisinopril, zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochloride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexyline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can comprise at least one compound of the invention, or pharmaceutically acceptable salts, solvates, esters or prodrugs thereof, and one or more antidiabetic medications. Useful antidiabetic medications include, but are not limited to, those useful for reducing blood glucose levels in a human. Other useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguanide (such as metformin and buformin), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

Mixtures of two, three, four or more of any of the pharmacological or therapeutic agents described above can be used in the compositions and therapeutic combinations of the present invention.

Since the present invention relates to treating conditions as discussed above, by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least one compound the invention, or a pharmaceutically acceptable salt, solvate, ester or prodrug thereof, and a separate pharmaceutical composition comprising at least one additional active agent such as those described herein. The kit may include directions for the administration of the separate components. The kit form is advantageous when, for example, the separate components must be administered in different dosage forms (e.g., oral and parenteral) or are administered at different dosage intervals.

In another embodiment, the present invention provides a method of treating, reducing, or ameliorating a disease or condition responsive to CB1 receptor antagonists. Non-limiting examples of such diseases or conditions include metabolic syndrome, obesity, excess waist circumference, excess adipose tissue, abnormal lipid profiles, insulin sensitivity, insulin resistance, neuroinflammatory disorders, cognitive disorders, psychosis, addictive behavior, gastrointestinal disorders, vascular conditions, hyperlipidaemia, dyslipidemia, atherosclerosis, hypercholesterolemia, hyperglycemia, sitosterolemia, vascular inflammation, stroke, diabetes (type 1 and type 2), cardiovascular conditions, and excess serum sterol(s) levels. Each such condition or disorder may be treated by administering, to a patient in need thereof, at least one compound (or composition) of the invention, or a pharmaceutically acceptable salt, solvate, ester or prodrug thereof, in an amount effective to treat the disease(s) or condition(s) being treated. In each such method, the at least one compound (or composition) of the invention may be administered alone or in combination with one or more additional active agents, such as one or more cholesterol lowering compounds, as described herein.

The treatment compositions and therapeutic combinations comprising at least one compound of the invention and at least one cholesterol lowering agent can be administered to inhibit the intestinal absorption of cholesterol in mammals. Such embodiments can be useful in the treatment and/or prevention of a variety of diseases, conditions, or disorders as are well known to those of ordinary skill in the art and include, for example vascular conditions, such as atherosclerosis, hypercholesterolemia and sitosterolemia, stroke, obesity and lowering of plasma levels of cholesterol in mammals, in particular in mammals.

In another embodiment of the present invention, the compounds, compositions, and therapeutic combinations of the present invention can be used to inhibit sterol or 5α-stanol absorption or to reduce plasma concentration of at least one sterol selected from the group consisting of phytosterols (such as sitosterol, campesterol, stigmasterol and avenosterol) and/or 5α-stanol (such as cholestanol, 5α-campestanol, 5α-sitostanol), cholesterol and mixtures thereof. Serum cholesterol levels, including LDL levels, can be reduced by administering to a mammal in need of such treatment an effective amount of at least one treatment composition or therapeutic combination comprising at least one selective CB1 receptor antagonist and at least one cholesterol lowering compound, for example a sterol absorption inhibitor described above. The reduction in plasma concentration of sterols or 5α-stanols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

The treatments of the present invention can also reduce the size or presence of plaque deposits in vascular vessels. The plaque volume can be measured using (IVUS), in which a tiny ultrasound probe is inserted into an artery to directly image and measure the size of atherosclerotic plaques, in a manner well know to those skilled in the art.

One or more compounds of the invention may also be administered in combination with a naturally occurring compound that acts to lower plasma cholesterol levels. Such naturally occurring compounds are commonly called nutraceuticals and include, for example, garlic extract, Hoodia plant extracts, and niacin.

The dosage of the additional therapeutic agent is generally dependent upon a number of factors including the health of the subject being treated, the extent of treatment desired, the nature and kind of concurrent therapy, if any, and the frequency of treatment and the nature of the effect desired. In one embodiment the dosage range of the additional therapeutic agent is in the range of from about 0.001 mg to about 100 mg per kilogram body weight of the individual per day. In another embodiment, the dosage range of the additional therapeutic agent is from about 0.1 mg to about 10 mg per kilogram body weight of the individual per day. However, some variability in the general dosage range may also be required depending upon the age and weight of the subject being treated, the intended route of administration, the particular additional therapeutic agent being administered and the like. The determination of dosage ranges and optimal dosages for a particular patient is also well within the ability of one of ordinary skill in the art.

According to the methods of the invention, one or more compounds the invention, or one or more compounds of the invention in combination with one or more additional therapeutic agents is administered to a subject in need of such treatment, for example in the form of a pharmaceutical composition. When one or more compounds of the invention is administered with one or more additional therapeutic agents, the compound of the present invention and at least one other therapeutic agent (e.g., anti-obesity agent, nicotine receptor partial agonist, dopaminergic agent, or opioid antagonist) may be administered either separately or in the pharmaceutical composition comprising both. In one embodiment, such administration is oral. In other embodiments, such administration is parenteral or transdermal.

When a combination of one or more compounds of the invention and at least one other therapeutic agent are administered together, such administration can be sequential in time or simultaneous. For sequential administration, one or more compounds of the invention and the additional therapeutic agent can be administered in any order. In one embodiment, such administration is oral. In another embodiment, such administration is oral and simultaneous. When one or more compounds of the invention and one or more additional therapeutic agents are administered sequentially, the administration of each can be by the same or by different methods.

In one embodiment, one or more compounds of the invention or a combination of one or more compounds of the invention and at least one additional therapeutic agent (referred to herein as a “combination”) is administered in the form of a pharmaceutical composition. Accordingly, one or more compounds of the invention or a combination can be administered to a patient separately or together in any conventional oral, rectal, transdermal, parenteral, (for example, intravenous, intramuscular, or subcutaneous) intracisternal, intravaginal, intraperitoneal, intravesical, local (for example, powder, ointment or drop), or buccal, or nasal, dosage form.

### EXAMPLES

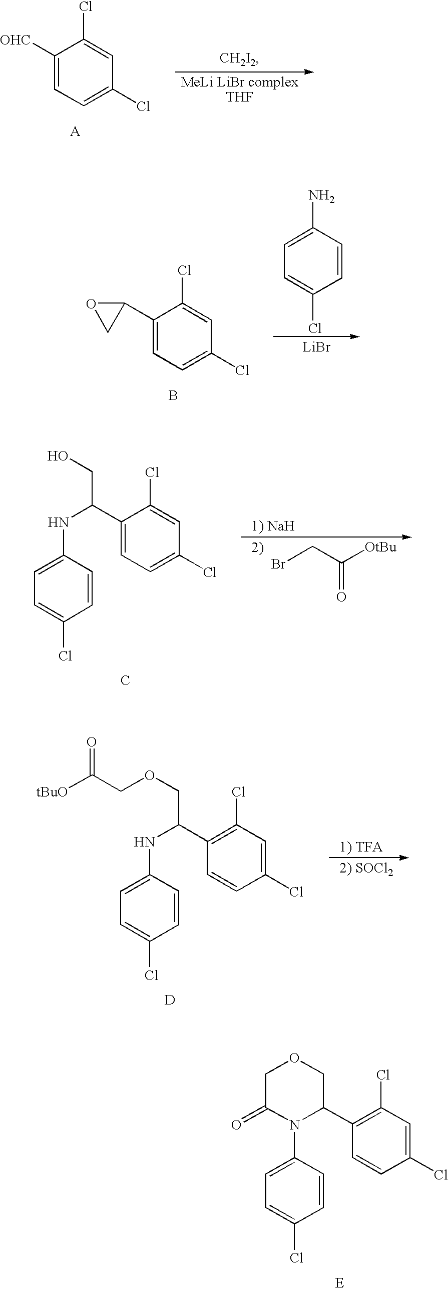

### Compound B:

To a solution of the aldehyde A (10.1 g, 58.1 mmol) in anhydrous THF (100 mL) was added diiodomethane (23.3 g, 87.2 mmol). The solution was cooled to 0° C. and MeLi.LiBr complex was added (1.5 M in hexanes, 77 mL, 116 mmol). The solution was stirred at 0° C. for 1 h followed by an additional 1 h at RT. The solution was poured into ice and extracted with EtOAc (3×). The combined organic layers were washed with brine, dried over Na₂SO₄, filtered and concentrated. The epoxide B was used without purification.

### Compound C:

To a flask containing the epoxide B (5.9 g, 31.2 mmol) was added 4-chloroaniline (3.98 g, 31.2 mmol) and LiBr (135 mg, 1.56 mmol). The flask was heated until all of the solid melted and the resultant oil was stirred at RT for 2.5 days. The crude material was purified directly via flash chromatography (SiO₂: gradient elution 100:0 to 65:35 hexanes:EtOAc) to afford the amino alcohol C (3.14 g) as a yellow oil.

### Compound D:

To a solution of the amino alcohol C (550 mg, 1.73 mmol) in anhydrous THF (20 mL) was added NaH (76 mg, 1.91 mmol). The mixture was stirred at RT for 1 h. To the mixture was added bromo-t-butyl acetate (373 mg, 1.91 mmol) and 18-crown-6 (114 mg, 0.43 mmol). The resultant solution was stirred at RT for 2.5 days. Water was added and the mixture was extracted with EtOAc (3×). The combined organic layers were washed with brine, dried over Na₂SO₄, filtered and concentrated. The crude material was purified via flash chromatography (SiO₂: gradient elution 100:0 to 75:25 hexanes:EtOAc) to afford the t-butyl ester D (380 mg) as a light yellow oil.

### Compound E:

Step 1:

To a solution of the t-butyl ester (380 mg, 0.88 mmol) in CH₂Cl₂ (3 mL) was added TFA (8 mL). The solution was stirred at RT for 2 days. To this solution was added CH₂Cl₂ followed by excess 2 M hydrogen chloride (in Et₂O). The solution was concentrated to afford the crude HCl salt.

Step 2: To the crude amino acid (0.88 mmol) from Step 1 in toluene (20 mL) was added pyridine (278 mg, 3.52 mmol). To this solution was added dropwise a solution of thionyl chloride (126 mg, 1.05 mmol) in toluene (10 mL) over 20 min. The solution was stirred at RT for 2 h. The solution was then heated to 50° C. for and stirred for an additional 2 h. The solution was cooled to RT and water was added. The mixture was extracted with EtOAc (3×). The combined organic layers were washed with brine, dried over Na₂SO₄, filtered and concentrated. The crude material was purified via flash chromatography (SiO₂: gradient elution 100:0 to 70:30 hexanes:EtOAc) to afford morpholinone E (160 mg) as a white solid.

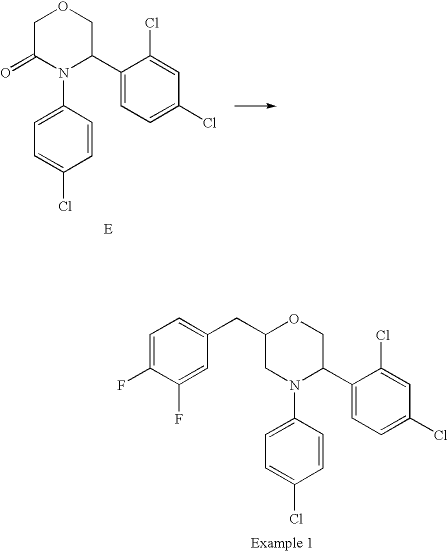

### Example 1

Step 1: To a solution of diisopropyl amine (143 mg, 1.41 mmol) in THF (10 mL) at 0° C. was added nBuLi (2 M in hexanes: 0.70 mL, 1.41 mmol). The solution was stirred at 0° C. for 30 min. The solution was then cooled to −78° C. To this solution was added a solution of the morpholinone E (386 mg, 1.08 mmol) in THF (10 mL). The resultant solution was stirred at −78° C. for 45 min. To this solution was added 3,4 difluorobenzyl bromide (268 mg, 1.30 mmol). The solution was stirred for 5 min at −78° C. followed by an additional 3 h at RT. Water was added to the solution and the mixture was extracted with EtOAc (3×). The combined organic layers were washed with brine, dried over Na₂SO₄, filtered and concentrated. The crude material was purified via flash chromatography (SiO₂: gradient elution 100:0 to 70:30 hexanes:EtOAc) to afford the alkylated product (80 mg).

Step 2: To a solution of the product from step 1 (80 mg, 0.17 mmol) in anhydrous THF (5 mL) was added borane THF complex (1 M in THF: 0.50 mL, 0.49 mmol). The solution was heated to reflux for 3 h. To the solution was slowly added 1 M HCl (aq.). The mixture was stirred at RT for 30 min. The mixture was then basified with saturated NaHCO₃ (aq.). The mixture was extracted with EtOAc (3×). The combined organic layers were washed with brine, dried over Na₂SO₄, filtered and concentrated. The crude material was purified via preparative thin layer chromatography (SiO₂: 9:1 hexanes:EtOAc) to afford Example 1.

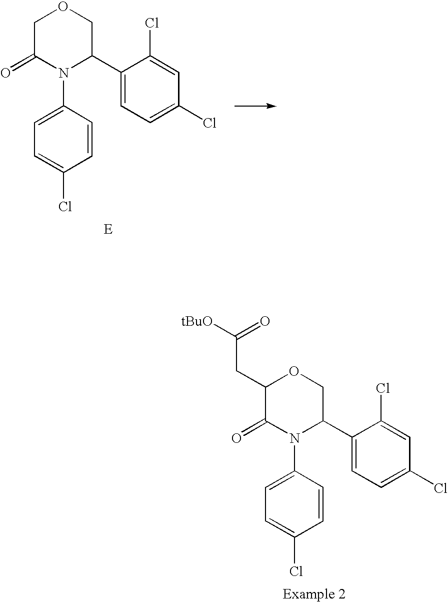

### Example 2

To a solution of E (230 mg, 0.64 mmol) in THF (10 mL) at −78° C. was added a solution of LHMDS (1 M in toluene: 0.77 mL, 0.77 mmol). The resultant solution was stirred at −78° C. for 45 min. To this solution was added bromo-t-butyl acetate (137 mg, 0.70 mmol) and the solution was stirred at −78° C. for 1 hr. Water was added and the mixture was allowed to warm to RT. The mixture was extracted with EtOAc (3×). The combined organic layers were washed with brine, dried over Na₂SO₄, filtered and concentrated. The crude material was purified via flash chromatography (SiO₂: gradient elution 100:0 to 60:40 hexanes:EtOAc) to afford Example 2 (165 mg) as a clear oil.

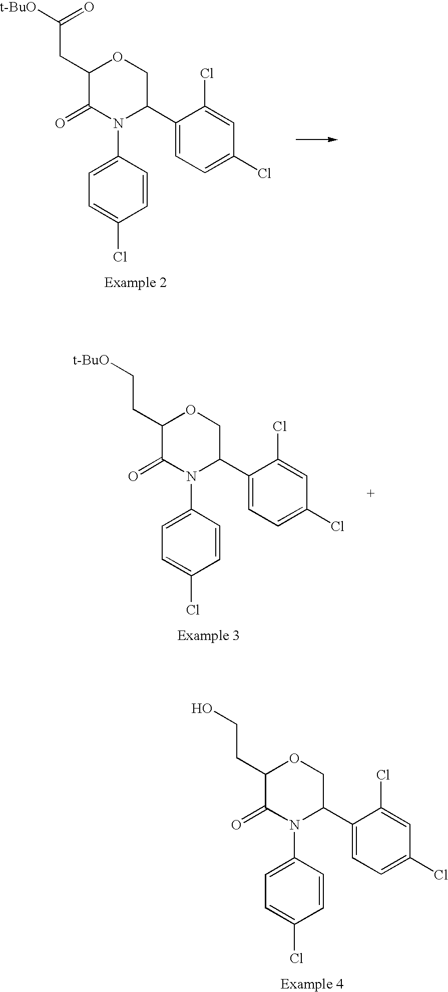

### Examples 3 and 4

To a solution of Example 2 (160 mg, 0.34 mmol) in THF (8 mL) was added borane.THF complex (1 M in THF: 1.70 mL, 1.70 mmol). The solution was heated to reflux for 16 h. To the solution was slowly added 1 M HCl (aq.). The mixture was stirred at RT for 30 min. The mixture was basified with saturated NaHCO₃ (aq.) and was extracted with EtOAc (3×). The combined organic layers were washed with brine, dried over Na₂SO₄, filtered and concentrated. The crude material was purified via flash chromatography (SiO₂: 9:1 hexanes:EtOAc) to afford Example 3 (40 mg) and Example 4 (64 mg).

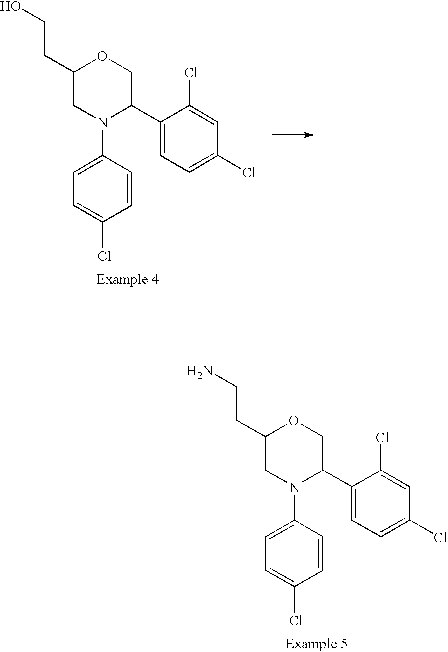

### Example 5

Step 1: To a solution of Example 4 (75 mg, 0.20 mmol) in THF (5 mL) was added PPh₃ (102 mg, 0.39 mmol) followed by DIAD (78 mg, 0.39 mmol). To this solution was added dropwise DPPA (107 mg, 0.39 mmol). The solution was stirred at RT overnight. The crude mixture was then concentrated and directly purified via flash chromatography (SiO₂: gradient elution: 100:0 to 70:30 hexanes:EtOAc) to afford the azide (55 mg).

Step 2: To a solution of the azide (55 mg, 0.13 mmol) in THF (5 mL) was added PPh₃ (70 mg, 0.27 mmol). The solution was heated to reflux for 4.5 h. Water (0.1 mL) was added and the mixture was heated to 50° C. for 2.5 days. The mixture was diluted with EtOAc, dried over Na₂SO₄, filtered and concentrated. The crude material was purified via preparative thin layer chromatography (SiO₂: 95:5:0.1 CH₂Cl₂: MeOH:7 N NH₃ in MeOH) to afford Example 5 (55 mg).

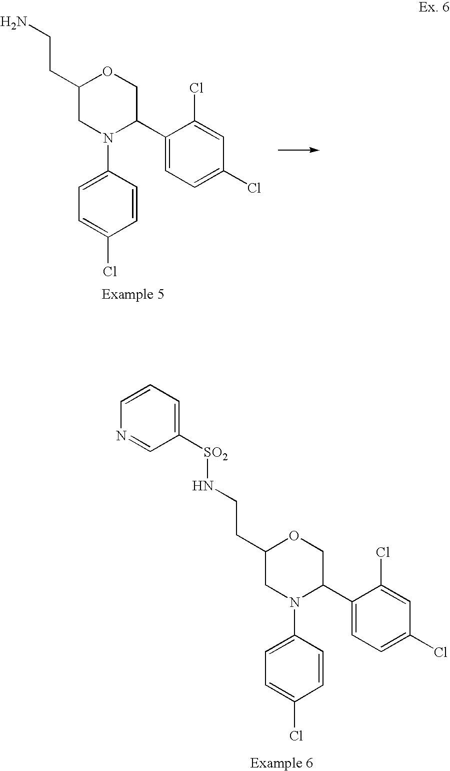

### Example 6

To a solution of Example 5 (16 mg, 0.042 mmol) in 1,2-dichloroethane (2 mL) was added diisopropylethyl amine (6.4 mg, 0.050 mmol) and 3-pyridine sulfonyl chloride hydrochloride (Combi-Blocks, San Diego, Calif.) (11 mg, 0.050 mmol). The resultant solution was stirred at RT overnight. The solution was diluted with CH₂Cl₂, washed with water, dried over Na₂SO₄, filtered and concentrated. The crude material was purified via preparative thin layer chromatography (SiO₂: 1:1 hexanes:EtOAc) to afford Example 6 (9 mg) as a clear oil.

### ASSAY

### Method for Evaluating Cannabinoid CB1 and CB2 Affinity

Competition binding assays for cannabinoid CB1 and CB2 affinity were performed by incubating commercially purchased membranes prepared from cells expressing each receptor subtype (8 μg pro) with 0.5 nM ³H—CP55,940, a non-selective cannabinoid agonist, along with concentrations of drug ranging from 0.0001-3 μM in Buffer A (5 mM MgCl₂, 2.5 mM EDTA and 013% BSA). Non-specific binding was defined in the presence of 10 μM CP55,940. For saturation studies, concentrations of ³H—CP55,940 ranging from 0.1-5 nM were incubated with membranes in the presence and absence of 10 μM CP55,940. Assays were terminated after incubation for 1½ hours by rapid filtration onto 0.3% polyethylenamine treated GF/C filterplates using a BRANDEL cell harvester. The plates were dried and MICROSCINT scintillation cocktail was added, after which the bound radioactivity was quantified using a TOPCOUNT scintillation counter.

The dissociation constant (K_{d}) of ³H—CP55,940 at the CB1 and CB2 receptor were determined by plotting specific binding at each concentration of radioligand, and analysis by non-linear regression. For competition studies, the concentration of each drug that inhibited 50 percent of ³H—CP55,940 binding (IC₅₀) was determined by non-linear regression analysis of the radioligand displacement curves. Affinity constants (K) were calculated using the equation derived by Cheng and Prusoff (1973), defined as: IC₅₀/1+[conc. ligand/K_{d}].

### GTPγS Binding Protocol

The functional efficacy of compounds to activate second messengers within the cell was determined utilizing the GTPγS binding assay. Guanine nucleotides are phosphorylated within the plasma membrane of the cell following binding and activation by agonists. A radiolabelled derivative of guanine triphosphate (GTP) is utilized in this assay as it cannot be dephosphorylated and therefore accumulates following agonist binding. The simultaneous presence of an antagonist into this system will shift the agonist concentration curve to the right, with increasing concentrations of antagonist producing a greater rightward shift in the dose-response curve of the agonist.

Commercially purchased membranes were incubated with 10 mM GDP to allow sufficient substrate for phosphorylation in the presence of agonist. The membranes were then pre-incubated with increasing concentrations of test compound for 30 minutes to determine if they were capable of stimulating phosphorylation alone. Increasing concentrations of the non-selective cannabinoid agonist WIN55,122 were then added in the presence or absence of each concentration of test compound. The assay was then incubated for 1 hour at room temperature. To complete the assay, ³⁵S-GTPγS was added and the assay incubated for another 30 minutes. Assays were terminated by rapid filtration onto 10 mM sodium phosphate-treated GF/C filterplates using a BRANDEL cell harvester. The plates were dried and Microscint scintillation cocktail was added, after which the bound radioactivity was quantified using a TOPCOUNT scintillation counter.

The stimulation of ³⁵S-GTPγS binding as a function of the concentration of the agonist WIN55,122, in the absence and presence of test compound, was plotted and the EC₅₀ determined by nonlinear regression analysis using GraphPad Prism software. A Schild analysis of the rightward shift in the dose response curve of WIN55,122 in the presence of test compound was determined by plotting the concentration of test compound against the negative log of the dose ratio [1−(EC₅₀ agonist+test compound/EC50 of agonist alone)]. A linear regression analysis yields the Kb, defined as the X-intercept of the linear equation.

In one embodiment, the compounds of the invention, and salts, solvates, or esters thereof, have Kᵢ values as measured in the above assay of about 800 nM or less (See Examples 2, 4 and 5). In another embodiment, the compounds of the invention, and salts, solvates, or esters thereof, have Kᵢ values of about 100 nM or less (See Example 1). In another embodiment, the compounds of the invention, and salts, solvates, or esters thereof, have Kᵢ values of about 55 nM or less (See Example 3). In another embodiment, the compounds of the invention, and salts, solvates, or esters thereof, have Kᵢ values of about 25 nM or less (See Example 6).

We claim:

1. A compound of Formula (I): 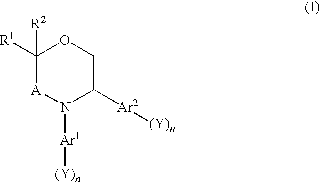
or a pharmaceutically acceptable salt, thereof wherein Ar¹, Ar², R¹, R², A, each Y, and each n is selected independently and wherein:
Ar¹ and Ar² are each independently aryl or heteroaryl;
R¹ is alkyl, —(C(R³)₂)ₘaryl, —(C(R³)₂)ₚOR⁴, —(C(R³)₂)ₚNR⁵R⁶, —(C(R³)₂)ₘheteroaryl, —(C(R³)₂)ₚC(O)OR⁴, —(C(R³)₂)ₚN₃, —(C(R³)₂)ₚS(O)₂R⁷, —(C(R³)₂)ₚC(O)R⁷, —(C(R³)₂)ₚS(O)₂N(R⁶)₂, —(C(R³)₂)ₚC(O)N(R⁴)₂, benzo-fused heterocycloalkyl or benzo-fused cycloalkyl, wherein each said aryl and each said heteroaryl of R¹ is optionally independently substituted with Z;
R² is H, alkyl, —(C(R³)₂)ₘaryl, —(C(R³)₂)ₚOR⁴ or —(C(R³)₂)ₚNR⁵R⁶;
A is —CH₂— or —C(O)—;
each Y is independently selected from the group consisting of halogen, CN, —OR⁴, alkyl, —C(O)N(R⁶)₂, —O-haloalkyl, —NR⁵R⁶, -alkyleneC(O)N(R⁶)₂, —C(O)Oalkyl, -alkyleneOR⁶, —S(O)₂R⁷, —C(O)R⁷, -alkyleneS(O)₂N(R⁶)₂, —S(O)₂N(R⁶)₂, cycloalkyl, heterocycloalkyl, haloalkyl, aryl, heteroaryl, —SR⁷, —O-Q-L-R⁹, —O-Q-S(O)₂N(R⁶)₂, —O-Q-C(O)N(R⁶)₂, —O-Q-N(R⁶)C(O)N(R⁶)₂;
each Q is a divalent radical independently selected from alkylene-, -alkenylene-, -alkynylene-, -cycloalkylene-, -heterocycloalkylene-, and -alkylene-cycloalkylene-;
each L is independently selected from —O—, —S—, —S(O)—, —S(O)₂—, —C(O)—, and —OC(O)—;
each n is independently 0-5;
each m is independently 0 to 5;
p is 1 to 5;
each R³ is independently selected from the group consisting of H, alkyl, cycloalkyl and —OR⁶;
each R⁴ is independently selected from the group consisting of H, alkyl, aryl, cycloalkyl, heteroaryl and -alkyleneOR⁶, wherein each said aryl and each said heteroaryl of R⁴ is optionally independently substituted with Z;
each R⁵ is independently selected from H, alkyl, heteroalkyl, aryl, heteroaryl, heterocycloalkyl, —S(O)₂R⁷, —C(O)R⁷, —C(O)N(R⁶)₂, —S(O)₂N(R⁶)₂, —C(O)N(R⁶)₂, and —C(O)OR⁷;
each R⁶ is independently selected from the group consisting of H, alkyl, heteroalkyl, aryl, cycloalkyl, heterocycloalkyl, and heteroaryl, wherein each said aryl and each said heteroaryl of R⁶ is optionally independently substituted with Z;
each R⁷ is alkyl, aryl, heteroalkyl, heteroaryl, cycloalkyl, haloalkyl, aralkyl, hydroxyalkyl, alkoxyalkyl, alkylene-N(R⁸)₂, heteroaralkyl or heterocycloalkyl, wherein each said aryl and each said heteroaryl of R⁷ is optionally independently substituted with Z;
each R⁸ is independently selected from the group consisting of H, alkyl; aryl, cycloalkyl and heteroaryl;
each Z is independently selected from halogen, alkyl, —OR⁶, —CN, -haloalkyl, —C(O)N(R⁶)₂, —NR⁵R⁶, -cycloalkyl, -alkyleneOR⁶, -alkyleneNR⁵R⁶, and -alkyleneC(O)N(R⁶)₂; and
each R⁹ is independently selected from H, alkyl, cycloalkyl, heterocycloalkyl, aryl, and heteroaryl, wherein each said aryl and each said heteroaryl of R⁹ is optionally independently substituted with Z.

2. A compound of claim 1 wherein A is —CH₂—.

3. A compound of claim 1 wherein A is —C(O)—.

4. A compound of claim 1 wherein R² is H.

5. A compound of claim 1 wherein Y is halogen.

6. A compound of claim 1 wherein Y is Cl.

7. A compound of claim 1 wherein n is 1 or 2.

8. A compound of claim 1 wherein each p is independently from 1 to 2.

9. (canceled)

10. A compound of claim 1 wherein R¹ is alkyl, —(C(R³)₂)₂aryl, —(C(R³)₂)₂OR⁴, —(C(R³)₂)₂NR⁵R⁶, —(C(R³)₂)₂heteroaryl, —(C(R³)₂)₂C(O)OR⁴ or —(C(R³)₂)₂C(O)N(R⁴)₂.

11. A compound of claim 1 wherein R² is H, alkyl, —(C(R³)₂)₂aryl, —(C(R³)₂)₂OR⁴, —(C(R³)₂)₂OR⁴ or —(C(R³)₂)₂NR⁵R⁶.

12. A compound of claim 1 wherein R¹ is —(CH₂)-aryl wherein said aryl is substituted with 1 to 5 halogens, —(CH₂)₂C(O)OR⁴, —(CH₂)₂OR⁴ or —(CH₂)₂NR⁵R⁶.

13. A compound of claim 1 wherein R¹ is

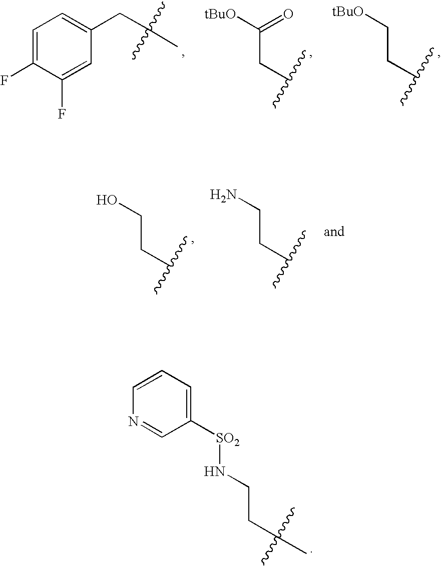

14.-18. (canceled)

19. A compound of claim 1 wherein R¹ is —(CH₂)₂C(O)OR⁴ or —(CH₂)₂OR⁴.

20. A compound of claim 1 having the structural Formula (I-A): 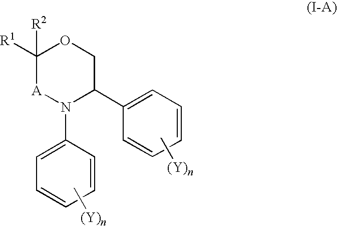
or a pharmaceutically acceptable salt thereof.

21. A compound of claim 1 having the structural Formula (I-B): 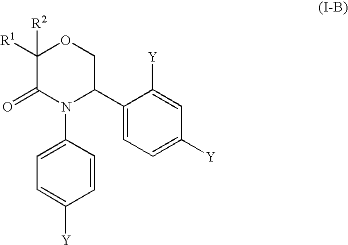
or a pharmaceutically acceptable salt thereof.

22. A compound of claim 1 having the structural Formula (I-C): 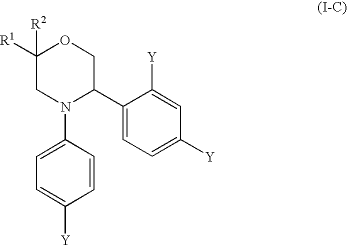
or a pharmaceutically acceptable salt thereof.

23. A compound of claim 22, wherein each Y is independently selected from halogen.

24. A compound of claim 23, wherein each Y is chlorine.

25. A compound of claim 24, wherein R² is H.

26. A compound of claim 1, or a pharmaceutically acceptable salt thereof, said compound being selected from the group consisting of:

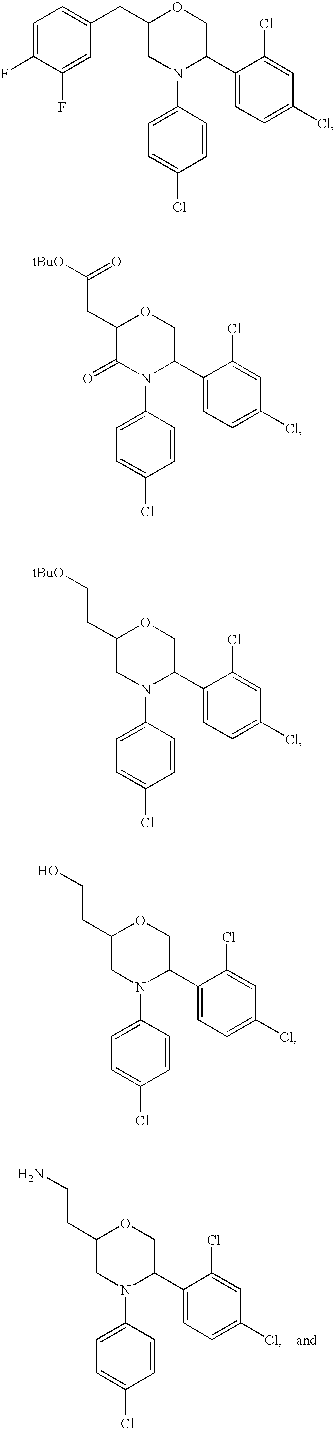

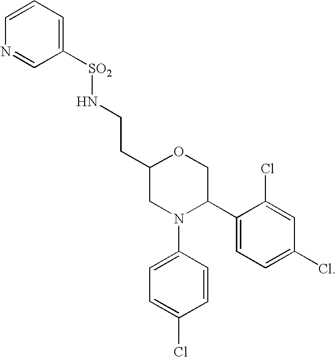

27.-31. (canceled)

32. A composition comprising a compound of claim 1, or a pharmaceutically acceptable salt thereof, and a pharmaceutically acceptable carrier.

33. A pharmaceutical composition comprising a compound of claim 1, or a pharmaceutically acceptable salt thereof, in combination with at least one additional therapeutic agent wherein said at least one additional therapeutic agent is selected from the group consisting of an antiobesity agent, an antidiabetic agent, and a lipid lowering agent.

34. (canceled)

35. A pharmaceutical composition of claim 33, wherein said antiobesity agent is selected from rimonabant, orlistat, sibutramine, bromocriptine, ephedrine, leptin, pseudoephedrine, and PYY₃₋₃₆;
said antidiabetic agent is selected from the group consisting of PPARγ agonist, PPARα/γ dual agonist, biguanidine, sulfonylurea, meglitinide, insulin, insulin secretagogue, and a dipeptidyl peptidase IV inhibitor; and
said lipid lowering agent is selected from the group consisting of a bile acid sequesterant, an HMG-CoA reductase inhibitor, a cholesterol absorption inhibitor, an ACAT inhibitor, a CETP inhibitor, a PPARα agonist, niacin and a niacin receptor agonist.

36. A pharmaceutical composition of claim 33 wherein said lipid lowering agent is a cholesterol absorption inhibitor.

37. (canceled)

38. The pharmaceutical composition of claim 36 wherein said sterol absorption inhibitor is ezetimibe.

39. A method of treating a disease, condition, or disorder responsive to CB1 receptor antagonists selected from the group consisting of metabolic syndrome, obesity, excess waist circumference, abnormal lipid profile, insulin resistance, a neuroinflammatory disorder, a cognitive disorder, a psychosis, an addictive behavior, a gastrointestinal disorder, and a cardiovascular condition, said method comprising administering to a patient in need thereof a therapeutically effective amount of at least one compound of claim 1, or a pharmaceutically acceptable salt thereof.

40. (canceled)

41. The method of claim 39, wherein said disease, disorder, or condition is metabolic syndrome.

42. A method of claim 39, further comprising administering at least one additional therapeutic agent selected from the group consisting of an antiobesity agent, an antidiabetic agent, or lipid lowering agent.

43. A method of claim 42, wherein:
said antiobesity agent is selected from the group consisting of rimonabant, orlistat, sibutramine, bromocriptine, ephedrine, leptin, pseudoephedrine, and PYY₃₋₃₆;
said antidiabetic agent is selected from the group consisting of PPARγ agonist, dual agonist, biguanidine, sulfonylurea, meglitinide, insulin, insulin secretagogue, and a dipeptidyl peptidase IV inhibitor; and
said lipid lowering agent is selected from the group consisting of a bile acid sequesterant, an HMG-CoA reductase inhibitor, a cholesterol absorption inhibitor, an ACAT inhibitor, a CETP inhibitor, a PPARα agonist, niacin and a niacin receptor agonist.

44. A method of claim 42 wherein said lipid lowering agent is a cholesterol absorption inhibitor.

45. (canceled)

46. A method of claim 42 wherein said sterol absorption inhibitor is ezetimibe.

47. (canceled)

48. A composition comprising:
at least one compound according to claim 1, or a pharmaceutically acceptable salt thereof, and
a pharmaceutically acceptable carrier.
